(19) United States
(12) Patent Application Publication
(10) Pub. No.: US 2017/0172493 A1
(43) Pub. Date: Jun. 22, 2017

(54) WEARABLE SYSTEM FOR PREDICTING ABOUT-TO-EAT MOMENTS

(71) Applicant: Microsoft Technology Licensing, LLC, Redmond, WA (US)

(72) Inventors: Tauhidur Rahman, Ithaca, NY (US); Mary Czerwinski, Kirkland, WA (US); Ran Gilad-Bachrach, Bellevue, WA (US); Paul R. Johns, Tacoma, WA (US); Asta Roseway, Clyde Hill, WA (US); Kael Robert Rowan, Kenmore, WA (US)

(21) Appl. No.: 14/973,645

(22) Filed: Dec. 17, 2015

(51) Int. Cl. A61B 5/00; G09B 19/00; A61B 5/0205; A63B 24/00

(52) U.S. Cl. CPC A61B 5/486; A63B 24/0062; G09B 19/0092; A61B 5/6801; A61B 5/6898; A61B 5/02055; A61B 5/7264; A61B 5/7405; A61B 5/742; A61B 5/7455; A61B 5/746; A61B 5/7275; A63B 2230/06; A63B 2230/50; A63B 2230/65; A63B 2220/40; A63B 2220/12; A63B 2220/17; A63B 2230/75; A63B 2220/30; A63B 2220/34

(57) ABSTRACT

A system is provided that predicts eating events for a user. The system includes a set of sensors each of which is configured to continuously measure a different physiological variable associated with the user and output a time-stamped data stream that includes the current value of this variable. A set of features is periodically extracted from the data stream output from each of the sensors, where these features have been determined to be specifically indicative of an about-to-eat moment. This set of features is then input into an about-to-eat moment classifier that has been trained to predict when the user is in an about-to-eat moment based on this set of features. Whenever an output of the classifier indicates that the user is currently in an about-to-eat moment, the user is notified with a just-in-time eating intervention.

### BACKGROUND

There is a prevalence of obesity across the globe that has become a major challenge to the world's healthcare systems and economies. For example, obesity is linked to many chronic diseases including diabetes, heart disease and cancer. A balanced diet and healthy eating habits (e.g., behaviors) are crucial to controlling obesity and maintaining good overall health. Since diet and health are closely related, dietary education and methods for maintaining awareness of one's own eating habits are and will continue to be universally important health topics. In fact, one of the cornerstones of modern public health policy today is to educate people across the globe about healthy dietary behaviors and encourage/motivate them to modify their eating habits accordingly.

### SUMMARY

Wearable system implementations described herein generally involve a system for predicting eating events for a user. In one exemplary implementation the system includes a set of mobile sensors, where each of the mobile sensors is configured to continuously measure a different physiological variable associated with the user and output a time-stamped data stream that includes the current value of this variable. For each of the mobile sensors, the data stream output from the mobile sensor is received, and a set of features is periodically extracted from this received data stream, where these features, which are among many features that can be extracted from this received data stream, have been determined to be specifically indicative of an about-to-eat moment. The set of features that is periodically extracted from the data stream received from each of the mobile sensors is then input into an about-to-eat moment classifier that has been trained to predict when the user is in an about-to-eat moment based on this set of features. Then, whenever an output of the classifier indicates that the user is currently in an about-to-eat moment, the user is notified with a just-in-time eating intervention. In another exemplary implementation the set of features that is periodically extracted from the data stream received from each of the mobile sensors is input into a regression-based time-to-next-eating-event predictor that has been trained to predict the time remaining until the onset of the next eating event for the user based on this set of features. Then, whenever an output of the predictor indicates that the current time remaining until the onset of the next eating event for the user is less than a prescribed threshold, the user is notified with a just-in-time eating intervention.

It should be noted that the foregoing Summary is provided to introduce a selection of concepts, in a simplified form, that are further described below in the Detailed Description. This Summary is not intended to identify key features or essential features of the claimed subject matter, nor is it intended to be used as an aid in determining the scope of the claimed subject matter. Its sole purpose is to present some concepts of the claimed subject matter in a simplified form as a prelude to the more-detailed description that is presented below.

### DESCRIPTION OF THE DRAWINGS

The specific features, aspects, and advantages of the wearable system implementations described herein will become better understood with regard to the following description, appended claims, and accompanying drawings where:

FIG. 1 is a diagram illustrating one implementation, in simplified form, of a system framework for realizing the wearable system implementations described herein.

FIG. 2 is a diagram illustrating another implementation, in simplified form, of a system framework for realizing the wearable system implementations described herein.

FIG. 3 is a flow diagram illustrating an exemplary implementation, in simplified form, of a process for predicting eating events for a user.

FIG. 4 is a flow diagram illustrating an exemplary implementation, in simplified form, of a process for training a machine-learned eating event predictor.

FIG. 5 is a diagram illustrating an exemplary implementation, in simplified form, of an eating event forecaster computer program for predicting eating events for a user.

FIG. 6 is a diagram illustrating an exemplary implementation, in simplified form, of an eating event prediction trainer computer program for training a machine-learned eating event predictor.

FIGS. 7 and 8 illustrate an exemplary set of time-stamped data streams, in simplified form, that is received from a set of mobile sensors each of which is configured to continuously measure a different physiological variable associated with a user and output a time-stamped data stream that includes the current value of this variable.

FIG. 9 is a flow diagram illustrating an exemplary implementation, in simplified form, of a process for periodically extracting a set of features from the time-stamped data stream that is received from each of the mobile sensors in the set of mobile sensors.

FIG. 10 is a diagram illustrating the estimated contributions of different feature groups in the training of a user-independent about-to-eat moment classifier to predict about-to-eat moments for any user.

FIG. 11 is a table illustrating the performance of different types of user-independent about-to-eat moment classifiers after they have been trained using the wearable system implementations described herein.

FIG. 12 is a graph illustrating how the performance of a TreeBagger type user-independent about-to-eat moment classifier changes as a uniform window length that is used for periodic feature extraction is changed.

FIG. 13 is a graph illustrating how the performance of the TreeBagger type user-independent about-to-eat moment classifier changes as the size of an about-to-eat definition window is changed.

FIG. 14 is a table illustrating the performance of different types of user-independent regression-based time-to-next-eating-event predictors after they have been trained using the wearable system implementations described herein.

FIG. 15 is a graph illustrating how the time remaining until the onset of the next eating event for a user that is predicted by a TreeBagger type user-independent regression-based time-to-next-eating-event predictor performs with respect to a ground truth reference.

FIG. 16 is a graph illustrating how the performance of the TreeBagger type user-independent regression-based time-to-next-eating-event predictor changes as the uniform window length that is used for periodic feature extraction is changed.

FIG. 17 is a diagram illustrating a simplified example of a general-purpose computer system on which various implementations and elements of the wearable system, as described herein, may be realized.

### DETAILED DESCRIPTION

In the following description of wearable system implementations reference is made to the accompanying drawings which form a part hereof, and in which are shown, by way of illustration, specific implementations in which the wearable system can be practiced. It is understood that other implementations can be utilized and structural changes can be made without departing from the scope of the wearable system implementations.

It is also noted that for the sake of clarity specific terminology will be resorted to in describing the wearable system implementations described herein and it is not intended for these implementations to be limited to the specific terms so chosen. Furthermore, it is to be understood that each specific term includes all its technical equivalents that operate in a broadly similar manner to achieve a similar purpose. Reference herein to “one implementation”, or “another implementation”, or an “exemplary implementation”, or an “alternate implementation”, or “one version”, or “another version”, or an “exemplary version”, or an “alternate version” means that a particular feature, a particular structure, or particular characteristics described in connection with the implementation or version can be included in at least one implementation of the wearable system. The appearances of the phrases “in one implementation”, “in another implementation”, “in an exemplary implementation”, “in an alternate implementation”, “in one version”, “in another version”, “in an exemplary version”, and “in an alternate version” in various places in the specification are not necessarily all referring to the same implementation or version, nor are separate or alternative implementations/versions mutually exclusive of other implementations/versions. Yet furthermore, the order of process flow representing one or more implementations or versions of the wearable system does not inherently indicate any particular order nor imply any limitations of the wearable system.

As utilized herein, the terms “component,” “system,” “client” and the like are intended to refer to a computer-related entity, either hardware, software (e.g., in execution), firmware, or a combination thereof. For example, a component can be a process running on a processor, an object, an executable, a program, a function, a library, a subroutine, a computer, or a combination of software and hardware. By way of illustration, both an application running on a server and the server can be a component. One or more components can reside within a process and a component can be localized on one computer and/or distributed between two or more computers. The term “processor” is generally understood to refer to a hardware component, such as a processing unit of a computer system.

Furthermore, to the extent that the terms “includes,” “including,” “has,” “contains,” variants thereof, and other similar words are used in either this detailed description or the claims, these terms are intended to be inclusive, in a manner similar to the term “comprising”, as an open transition word without precluding any additional or other elements.

### 1.0 Introduction

This section introduces several different concepts, in simplified form, that are employed in the more-detailed description of the wearable system implementations that is presented below.

As is appreciated in the health sciences and the art of human biology, eating is one of the most fundamental yet complex biological processes of the human body. A person's eating habits (e.g., behaviors) play a primary role in determining their health, wellness and happiness. Irregular eating habits and disproportionate or inadequate dietary behaviors may increase the likelihood of severe health issues such as obesity. As described heretofore, there is a prevalence of obesity across the globe. More particularly, according to the World Health Organization more than 1.9 billion adults (age 18 and older) across the globe were overweight in 2014. In the United States, two out of every three adults is considered to be overweight or obese. This prevalence of obesity has become a major challenge to the world's healthcare systems and economies. For example, obesity is a leading cause of preventable death second only to smoking. In summary, obesity is a grave issue that faces the entire globe.

As is appreciated in the arts of behavioral modification and behavioral intervention technologies, an intervention is most effective when it occurs just before a person starts to perform an activity that the intervention is intended to either prevent from happening or curtail—such an intervention is sometimes referred to as a just-in-time intervention. Previous research studies in a variety of health domains have found that just-in-time interventions are maximally effective in encouraging and motivating the desired behavior change since they prompt the person at a critical point of decision (e.g., just before the person begins the behavior that is desired to change). In many health domains just-in-time interventions are triggered upon detecting certain events or conditions which are commonly a precursor of a negative health outcome. Such moments of high risk or heightened vulnerability, when coupled with a person's ineffective coping response, may easily lead the person toward decreased self-efficacy and possibly to relapse. Researchers working in the areas of alcohol addiction, drug addiction, smoking addiction, and stress management use these high risk and heightened vulnerability moments as optimally opportune moments for triggering just-in-time patient interventions since the patient gets the chance to cope, divert or circumvent the behavior which constitutes the negative health outcome before they begin the behavior. Research has also shown that the patient is often especially receptive to an intervention strategy during these high risk and heightened vulnerability moments.

As is also appreciated in the health sciences and the art of human biology, one of the fundamental causes of obesity is the over-consumption of food by many people. With particular regard to people's eating habits, previous research studies have also found that adults consume about 92 percent of the food that is served to them irrespective of their perceived self-control, current emotional state, and other external variables. This finding suggests that just-in-time eating interventions would be maximally effective in changing a person's eating habits toward better and healthier eating behavior since such interventions occur just prior to the person's actual eating events—support for this assertion can be found in the intuition that after a person has decided to have a cookie and perhaps has already had a bite of the cookie, it is much more difficult for the person to stop eating the cookie.

### 2.0 Wearable System for Predicting About-To-Eat Moments

The term “eating event” is used herein to refer to a given finite period time in a person's life during which the person eats one or more types of food. Exemplary types of eating events include breakfast, brunch, lunch, dinner, and a snack. The term “about-to-eat moment” is used herein to refer to the moment (e.g., the temporal episode) in a person's life just before the person begins a new eating event. In other words, an about-to-eat moment is a certain period of time that immediately precedes when a person starts to eat—this period of time is hereafter referred to as an about-to-eat definition window. It is noted that the about-to-eat definition window can have various values. By way of example but not limitation, in a tested implementation of the wearable system described herein the about-to-eat definition window was set to be 30 minutes. The term “user” is used herein to refer to a person who is using the wearable system implementations described herein.

The wearable system implementations described herein are generally applicable to the task of automatically predicting a user's eating events. In other words, rather than simply detecting when a user is currently eating, the wearable system implementations can be utilized to predict the user's next eating event ahead of time (e.g., a prescribed period of time before the onset (e.g., the beginning/start) of the next eating event for the user), thus providing the user with an opportunity to modify their behavior and choose not to begin/start the eating event. More particularly and as will be described in more detail hereafter, in one implementation of the wearable system a user's about-to-eat moments are predicted and the user may be automatically notified about such moments with a just-in-time eating intervention. In another implementation of the wearable system the current time remaining until the onset of the next eating event for a user is predicted and whenever this time is less than a prescribed threshold, the user may be automatically notified with a just-in-time eating intervention.

The wearable system implementations described herein are advantageous for various reasons including, but not limited to, the following. As will be appreciated from the more-detailed description that follows, the wearable system implementations can be used to encourage/motivate healthy eating habits in users (e.g., the wearable system implementations can nudge users towards healthy eating decision making). The wearable system implementations are also noninvasive and produce accurate results (e.g., can accurately predict users' eating events) for users having a wide variety of eating styles. The wearable system implementations are also context-aware since they adapt their behavior based on current information that is continually sensed from a given user and their environment. The wearable system implementations also discretely communicate each eating event prediction to each user, and thus address the privacy concerns of many people who are looking to either lose weight or modify their eating habits.

As described heretofore, the just-in-time eating interventions that are provided to users of the wearable system implementations described herein are maximally effective in encouraging and motivating the users to change their eating habits toward better and healthier eating behavior. The wearable system implementations are also easy to use and consume very little of the users' time and attention (e.g., the wearable system implementations require a very low level of user engagement). For example, the wearable system implementations eliminate the need for users to have to utilize various conventional manual food journaling methods (such as pen and paper, or a mobile software application, among others) in order to painstakingly log everything they eat throughout each day. The wearable system implementations also succinctly communicate each eating event prediction to each user without presenting the user with excessive and irrelevant information. Accordingly, users are prone to utilize the wearable system implementations on an ongoing basis, even after the novelty of these implementations fades.

### 2.1 System and Process Framework

This section describes different exemplary implementations of a system framework and a process framework that can be used to realize the wearable system implementations described herein. It is noted that in addition to the system framework and process framework implementations described in this section, various other system framework and process framework implementations may also be used to realize the wearable system implementations.

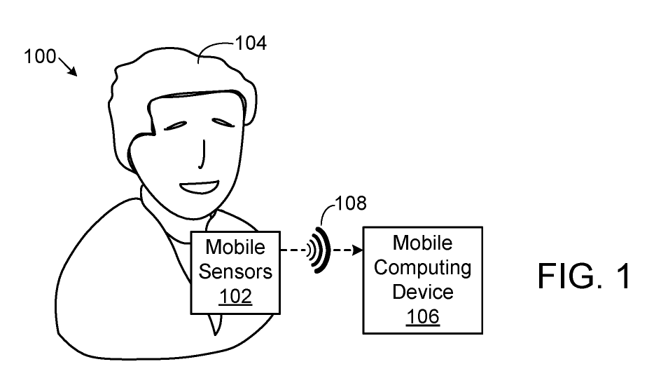

FIG. 1 illustrates one implementation, in simplified form, of a system framework for realizing the wearable system implementations described herein. As exemplified in FIG. 1, the system framework 100 includes a set of mobile (e.g., portable) sensors 102 each of which is either physically attached to (e.g., worn on) the body of, or carried by, a user 104 as they go about their day. As will be appreciated from the more detailed description that follows, the set of mobile sensors 102 is multi-modal in that each of the mobile sensors 102 is configured to continuously (e.g., on an ongoing basis) and passively measure (e.g., capture) a different physiological variable associated with the user 104 as they go about their day, and output a time-stamped data stream that includes the current value of this variable. In other words, the set of mobile sensors 102 continuously collect various types of information related to the user's 104 current physiology and their different eating events. Exemplary types of mobile sensors 102 that may be employed in the wearable system implementations are described in more detail hereafter.

Referring again to FIG. 1, the system framework 100 also includes a conventional mobile computing device 106 that is carried by the user 104. In an exemplary implementation of the wearable system described herein the mobile computing device is either a conventional smartphone or a conventional tablet computer. Each of the mobile sensors 102 is configured to wirelessly transmit 108 the time-stamped data stream output from the sensor to the mobile computing device 106. The mobile computing device 106 is according configured to wirelessly receive 108 the various data streams transmitted from the set of mobile sensors 102. The wireless communication 108 of the various data streams output from the set of mobile sensors 102 can be realized using various wireless technologies. For example, in a tested version of the wearable system implementations described herein this wireless communication 108 was realized using a conventional Bluetooth personal area network. Another version of the wearable system implementations is possible where the wireless communication 108 is realized using a conventional Wi-Fi local area network. Yet another version of the wearable system implementations is also possible where the wireless communication 108 is realized using a combination of different wireless networking technologies.

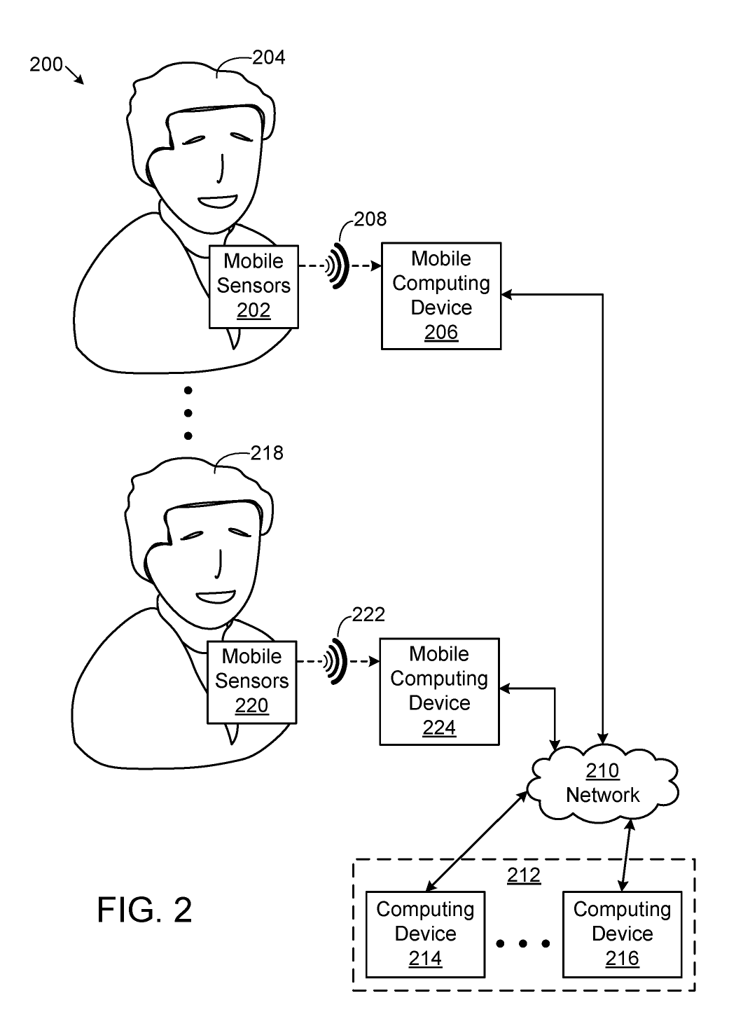

FIG. 2 illustrates another implementation, in simplified form, of a system framework for realizing the wearable system implementations described herein. As exemplified in FIG. 2, the system framework 200 includes the aforementioned set of mobile sensors 202/220 each of which is either physically attached to the body of, or carried by, each of one or more users 204/218 as they go about their day. The system framework 200 also includes the aforementioned mobile computing device 206/224 that is carried by each of the users 204/218, and is configured to wirelessly receive 208/222 the various time-stamped data streams transmitted from the set of mobile sensors 202/220. The mobile computing device 206/224 is further configured to communicate over a data communication network 210 such as the Internet (among other types of networks) to a cloud service 212 that operates on one or more other computing devices 214/216 that are remotely located from the mobile computing device 206/224. The remote computing devices 214/216 can also communicate with each other via the network 210. The term “cloud service” is used herein to refer to a web application that operates in the cloud and can be hosted on (e.g., deployed at) a plurality of data centers that can be located in different geographic regions (e.g., different regions of the world).

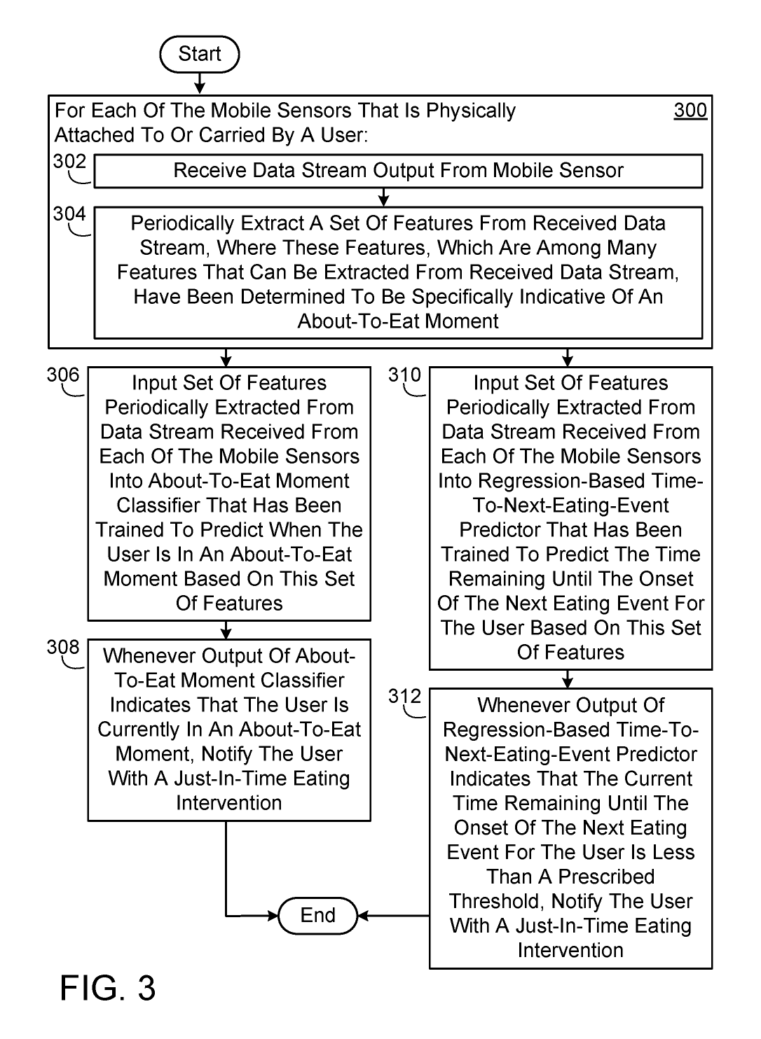

FIG. 3 illustrates an exemplary implementation, in simplified form, of a process for predicting eating events for a user. As exemplified in FIG. 3, the process starts with the following actions taking place for each of the mobile sensors that is either physically attached to the body of, or carried by, the user as they go about their day (process action 300). First, the data stream that is output from the mobile sensor is received (process action 302). A set of features is then periodically extracted from this received data stream, where these features, which are among many features that can be extracted from this received data stream, have been determined to be specifically indicative of an about-to-eat moment (process action 304). Exemplary methods for performing this periodic feature extraction and exemplary types of features that may be periodically extracted are described in more detail hereafter. In one implementation of the wearable system described herein the set of features that is periodically extracted from the data stream received from each of the mobile sensors is then input into an about-to-eat moment classifier that has been trained to predict when the user is in an about-to-eat moment based on this set of features (process action 306). In other words, the about-to-eat moment classifier has been trained to predict when an eating event for the user is about to occur (e.g., expected to occur within the aforementioned about-to-eat definition window). This classifier training is described in more detail hereafter.

The wearable system implementations described herein can train various types of classifiers. By way of example but not limitation, in one implementation of the wearable system described herein the classifier that is trained is a conventional linear type classifier. In another implementation of the wearable system the classifier that is trained is a conventional reduced error pruning (also known as a REPTree) type classifier. In another implementation of the wearable system the classifier that is trained is a conventional support vector machine type classifier. In another implementation of the wearable system the classifier that is trained is a conventional TreeBagger type classifier. Referring again to FIG. 3, whenever an output of the about-to-eat moment classifier indicates that the user is currently in an about-to-eat moment (e.g., an eating event for the user is about to occur), the user is automatically notified with a just-in-time eating intervention (process action 308). This notification can be provided to the user in various ways. By way of example but not limitation, the user notification may include a message that is displayed on a display screen of the mobile computing device that is carried by the user. The user notification may also include an audible alert that is output from the mobile computing device. The user notification may also include a haptic alert that is output from the mobile computing device. Exemplary types of just-in-time eating interventions are described in more detail hereafter.

The automatic generation of a just-in-time eating intervention for the user advantageously maximizes the usability of the mobile computing device that is carried by the user in various ways. For example and as described heretofore, the user does not have to run a food journaling application on their mobile computing device and painstakingly log everything they eat into this application. Additionally, the intervention is succinct and does not present the user with excessive and irrelevant information. As such, the automatically generated just-in-time eating intervention advantageously maximizes the efficiency of the user when they are using their mobile computing device.

Referring again to FIG. 3, in another implementation of the wearable system described herein the set of features that is periodically extracted from the data stream received from each of the mobile sensors is input into a regression-based time-to-next-eating-event predictor that has been trained to predict the time remaining until the onset of the next eating event for the user based on this set of features (process action 310). This predictor training is described in more detail hereafter. The wearable system implementations described herein can train various types of predictors. By way of example but not limitation, in one implementation of the wearable system the predictor that is trained is a conventional linear type predictor. In another implementation of the wearable system the predictor that is trained is a conventional reduced error pruning type predictor. In another implementation of the wearable system the predictor that is trained is a conventional sequential minimal optimization type predictor. In another implementation of the wearable system the predictor that is trained is a conventional TreeBagger type predictor.

Referring again to FIG. 3, whenever an output of the regression-based time-to-next-eating-event predictor indicates that the current time remaining until the onset of the next eating event for the user is less than a prescribed time threshold, the user is automatically notified with a just-in-time eating intervention (process action 312). This notification can be provided to the user in the various ways described heretofore. In a tested implementation of the wearable system described herein the just-described time threshold was set to be 30 minutes.

The just-in-time eating intervention described herein can include various types of information that welcomes a positive eating behavior. By way of example but not limitation, in one implementation of the wearable system described herein the just-in-time eating intervention may include diet-related information such as reminding the user to eat a balanced meal, or reminding the user of their calories allowance, or the like. In another implementation of the wearable system the just-in-time eating intervention may suggest a different timing for when the user eats again. In yet another implementation of the wearable system the just-in-time eating intervention may be customized/personalized by the user to meeting their particular needs/desires. In yet another implementation of the wearable system the just-in-time eating intervention may be generated using the conventional PopTherapy micro-intervention method (e.g., the just-in-time eating intervention may include a text prompt that tells the user what to do and a URL (Uniform Resource Locator) that when selected by the user launches a prescribed web site application that provides an appropriate micro-intervention).

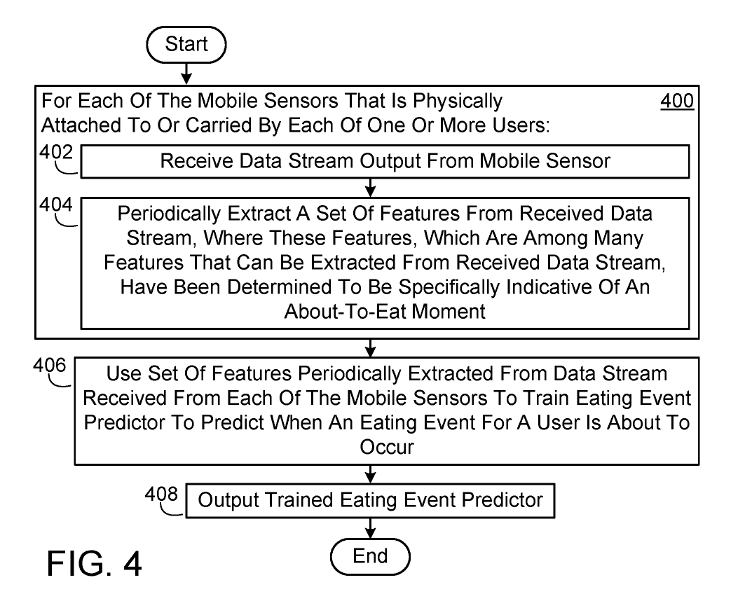

FIG. 4 illustrates an exemplary implementation, in simplified form, of a process for training a machine-learned eating event predictor. As exemplified in FIG. 4, the process starts with the following actions taking place for each of the mobile sensors that is either physically attached to the body of, or carried by, each of one or more users as they go about their day (process action 400). First, the data stream that is output from the mobile sensor is received (process action 402). The aforementioned set of features is then periodically extracted from this received data stream (process action 404). The set of features that is periodically extracted from the data stream received from each of the mobile sensors is then used to train the predictor to predict when an eating event for a user is about to occur (process action 406). The trained predictor is then output (process action 408). As will be described in more detail hereafter, in one implementation of the wearable system described herein the set of features that is periodically extracted from the data stream received from each of the mobile sensors is selected such that the trained predictor is user-independent and as such may be utilized to predict when an eating event for any user is about to occur. In a tested implementation of the wearable system the set of mobile sensors was physically attached to the body of, or carried by, each of eight different users (three female and five male) ranging in age from 26 to 54 years, and data streams were received from the set of mobile sensors for a period of five days. An alternate implementation of the wearable system is also possible where the set of features that is periodically extracted from the data stream received from each of the mobile sensors is selected such that the trained predictor is user-dependent.

In one implementation of the wearable system described herein the machine-learned eating event predictor is the aforementioned about-to-eat moment classifier that is trained to predict when a user is in an about-to-eat moment. In another implementation of the wearable system the machine-learned eating event predictor is the aforementioned regression-based time-to-next-eating-event predictor. Referring again to FIG. 4, in this particular implementation the action of periodically extracting a set of features from the data stream received from each of the mobile sensors (action 404) includes the action of mapping each of the features in the set of features that is periodically extracted from this received data stream to the current time remaining until the next eating event, where this current time remaining is determined by analyzing the data stream received from each of the mobile sensors. Additionally, the action of using the set of features that is periodically extracted from the data stream received from each of the mobile sensors to train the predictor to predict when an eating event for a user is about to occur (action 406) includes the action of using the set of features that is periodically extracted from the data stream received from each of the mobile sensors in combination with the just-described mapping of each of the features in this set of features to train the time-to-next-eating-event predictor to predict the time remaining until the onset of the next eating event for the user.

Referring again to FIG. 4, in an alternate implementation of the wearable system described herein the action of using the set of features that is periodically extracted from the data stream received from each of the mobile sensors to train the predictor to predict when an eating event for a user is about to occur (action 406) may be implemented as follows. First, the set of features that is periodically extracted from the data stream received from each of the mobile sensors may be input into an overall set of features. A combination of a conventional correlation-based feature selection method and a conventional best-first decision tree machine learning method may then be used to select a subset of the features in the overall set of features. This selected subset of the features may then be used to train the predictor to predict when an eating event for a user is about to occur. As is appreciated in the art of machine learning, the correlation-based feature selection method is based on the central hypothesis that a good feature set contains features that are highly correlated with the target class, but are uncorrelated with each other. Accordingly, the correlation-based feature selection method evaluates the “goodness” of each of the features in the overall set of features based on two criteria, namely, whether or not the feature is highly indicative of the target class, and whether or not the feature is highly uncorrelated with the features that have already been selected from the overall set of features. In other words, the correlation-based feature selection method selects features from the overall set of features that are highly indicative of the target class, and are highly uncorrelated with the features that have already been selected from the overall set of features.

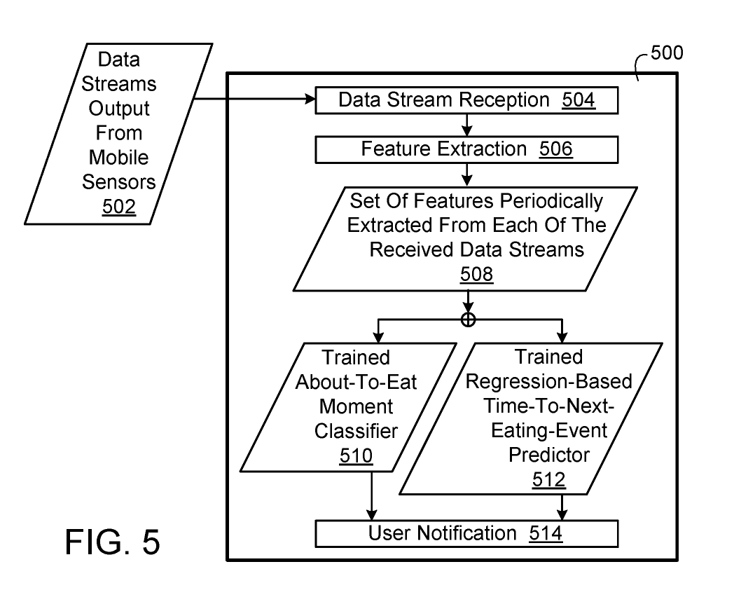

FIG. 5 illustrates an exemplary implementation, in simplified form, of an eating event forecaster computer program for predicting eating events for a user. As exemplified in FIG. 5, the eating event forecaster computer program 500 includes a data stream reception sub-program 504, a feature extraction sub-program 506, and a user notification sub-program 514. Each of these sub-programs 504/506/514 is realized on a computing device such as that which is described in more detail in the Exemplary Operating Environments section which follows. More particularly and by way of example but not limitation, in one implementation of the wearable system described herein the sub-programs 504/506/514 may all be realized on the mobile computing device that is carried by the user. In another implementation of the wearable system one or more of the sub-programs 504/506/514 may be realized on the mobile computing device and the other sub-programs may be realized on the aforementioned other computing devices that are remotely located from the mobile computing device.

Referring again to FIG. 5, the data stream reception sub-program 504 receives the data streams that are output from the mobile sensors 502. The feature extraction sub-program 506 periodically extracts the aforementioned set of features 508 from each of the received data streams and either inputs this set of features 508 into an about-to-eat moment classifier 510 that has been trained to predict when the user is in an about-to-eat moment based on the set of features 508, or inputs the set of features 508 into a regression-based time-to-next-eating-event predictor 512 that has been trained to predict the time remaining until the onset of the next eating event for the user based on the set of features 508. Whenever an output of the classifier 510 indicates that the user is currently in an about-to-eat moment, the user notification sub-program 514 notifies the user with a just-in-time eating intervention. Whenever an output of the predictor 512 indicates that the current time remaining until the onset of the next eating event for the user is less than the aforementioned prescribed time threshold, the user is notified with a just-in-time eating intervention.

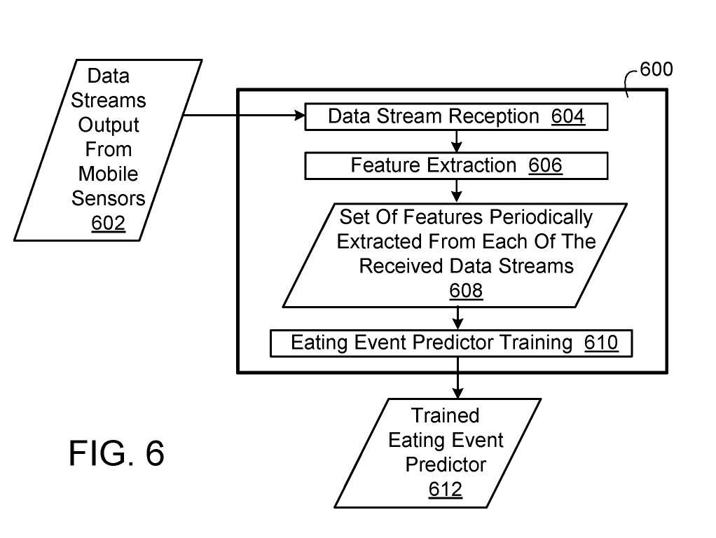

FIG. 6 illustrates an exemplary implementation, in simplified form, of an eating event prediction trainer computer program for training a machine-learned eating event predictor. As exemplified in FIG. 6, the eating event prediction trainer computer program 600 includes a data stream reception sub-program 604, a feature extraction sub-program 606, and an eating event predictor training sub-program 610. Each of these sub-programs 604/606/610 is realized on a computing device such as that which is described in more detail in the Exemplary Operating Environments section which follows. More particularly and by way of example but not limitation, in one implementation of the wearable system described herein the sub-programs 604/606/610 may all be realized on the mobile computing device that is carried by the user. In another implementation of the wearable system one or more of the sub-programs 604/606/610 may be realized on the mobile computing device and the other sub-programs may be realized on the other computing devices that are remotely located from the mobile computing device.

Referring again to FIG. 6, the data stream reception sub-program 604 receives the data streams that are output from the mobile sensors 602. The feature extraction sub-program 606 periodically extracts the set of features 608 from each of the received data streams. The eating event predictor training sub-program 610 uses this set of features 608 to train the machine-learned eating event predictor to predict when an eating event for a user is about to occur. After this training has been completed the eating event predictor training sub-program 610 outputs the trained eating event predictor 612.

### 2.2 User Data Collection

As described heretofore, the wearable system implementations described herein employ a multi-modal set of mobile sensors each of which is either physically attached to the body of, or carried by, a user. Each of the mobile sensors is configured to continuously and passively measure a different physiological variable associated with the user as they go about their day, and output a time-stamped data stream that includes the current value of this variable. The wearable system implementations can employ one or more of a wide variety of different types of mobile sensor technologies. For example, the set of mobile sensors may include a conventional heart rate sensor that outputs a data stream which includes the current heart rate of the user whose body the heart rate sensor is attached to. The set of mobile sensors may also include a conventional skin temperature sensor that outputs a data stream which includes the current skin temperature of the user whose body the skin temperature sensor is attached to. The set of mobile sensors may also include a conventional 3-axis accelerometer that outputs a data stream which includes the current three-dimensional (3D) linear velocity of the user whose body the accelerometer is attached to, or who is carrying the accelerometer. The set of mobile sensors may also include a conventional gyroscope that outputs a data stream which includes the current 3D angular velocity of the user whose body is gyroscope is attached to, or who is carrying the gyroscope. The set of mobile sensors may also include a conventional global positioning system (GPS) sensor that outputs a data stream which includes the current longitude of the user whose body the GPS sensor is attached to, or who is carrying the GPS sensor, and also outputs another data stream that includes the current latitude of this user. As is appreciated in the art of global positioning, the combination of the user's current longitude and latitude define the user's current physical location.

The set of mobile sensors may also include a conventional electrodermal activity sensor that outputs a data stream which includes the current electrodermal activity of a user whose body the electrodermal activity sensor is physically attached to. As is appreciated in the art of emotion analytics, electrodermal activity refers to electrical changes measured at the surface of a person's skin that arise when the skin receives innervating signals from the person's brain. For most people, when they experience emotional arousal, increased cognitive workload, or physical exertion, their brain sends signals to their skin to increase their level of sweating, which increases their skin's electrical conductance in a measurably significant way. As such, a person's electrodermal activity is a good indicator of their level of psychological arousal. In a tested version of the wearable system implementations described herein the conventional Q sensor manufactured by Affectiva, Inc. was used for the electrodermal activity sensor. However, it is noted that the wearable system implementations also support the use of any other type of electrodermal activity sensor.

The set of mobile sensors may also include a conventional body conduction microphone (also referred to as a bone conduction microphone) that outputs a data stream which includes current non-speech body sounds that are conducted through the body surface of a user whose body the body conduction microphone is physically attached to. In an exemplary implementation of the wearable system described herein the body conduction microphone was directly attached to the user's skin in the laryngopharynx region of the user's neck. In a tested version of the wearable system implementations described herein the conventional BodyBeat piezoelectric-sensor-based microphone was used for the body conduction microphone—this particular microphone captures a diverse range of non-speech body sounds (e.g., chewing and swallowing (among other sounds of food intake), breath, laughter, cough, and the like). However, it is noted that the wearable system implementations also support the use of any other type of body conduction microphone.

The set of mobile sensors may also include a conventional wearable computing device that provides health and fitness tracking functionality, and outputs one or more time-stamped data streams each of which includes the current value of a different physiological variable associated with a user whose body the wearable computing device is physically attached to. For simplicity sake such a wearable computing device is hereafter referred to as a health/fitness tracking device. It will be appreciated that one or more of the aforementioned different types of mobile sensors is integrated into the health/fitness tracking device. In a tested implementation of the wearable system described herein the health/fitness tracking device was directly attached to the user's wrist. It is noted that many different types of health/fitness tracking devices are commercially available today. By way of example but not limitation, in a tested version of the wearable system implementations described herein the conventional Microsoft Band was used for the health/fitness tracking device. In an exemplary implementation of the wearable system the health/fitness tracking device outputs a data stream that includes a current cumulative value for the step count of the user. The wearable computing device also outputs a data stream that includes a current cumulative value for the calorie expenditure of the user. The wearable computing device also outputs a data stream that includes the current speed of movement of the part of the user's body to which the wearable computing device is attached. For example, in the aforementioned tested implementation where the wearable computing device was attached to the user's wrist, this data stream includes the current speed of movement of the user's arm.

The set of mobile sensors may also include the aforementioned mobile computing device that is carried by a user, and outputs one or more time-stamped data streams each of which includes the current value of a different physiological variable associated with the user. In an exemplary implementation of the wearable system described herein the mobile computing device includes an application that runs thereon and allows the user to manually enter/log (e.g., self-report) various types of information corresponding to each of their actual eating events. In a tested implementation of the wearable system this application allowed the user to self-report when they begin a given eating event, their affect (e.g., their emotional state) and stress level at the beginning of the eating event, the intensity of their craving and hunger at the beginning of the eating event, the type of meal they consumed during the eating event, the amount of food and the “healthiness” of the food they consumed during the eating event, when they end the eating event, their affect and stress level at the end of the eating event, and their level of satisfaction/satiation at the end of the eating event. In an exemplary realization of this tested implementation the user reported their affect using the conventional Photographic Affect Meter tool; the user reported their stress level, the intensity of their craving and hunger, the amount of food they consumed, the healthiness of the food they consumed, and their level of satisfaction/satiation using a numeric scale (e.g., one to seven). The mobile computing device outputs a data stream that includes this self-reported information.

The mobile computing device that is carried by a user may also output a data stream that includes the current network location of the mobile computing device. As is appreciated in the art of wireless networking, the current network location of the mobile computing device may be used to approximate the user's current physical location in the case where the data streams that include the current longitude and current latitude of the user are not currently available. The current network location of the mobile computing device can be determined using various conventional methods. For example, the current network location of the mobile computing device can be determined by performing multilateration or triangulation between cell phone towers having known physical locations, or between Wi-Fi base stations having known physical locations.

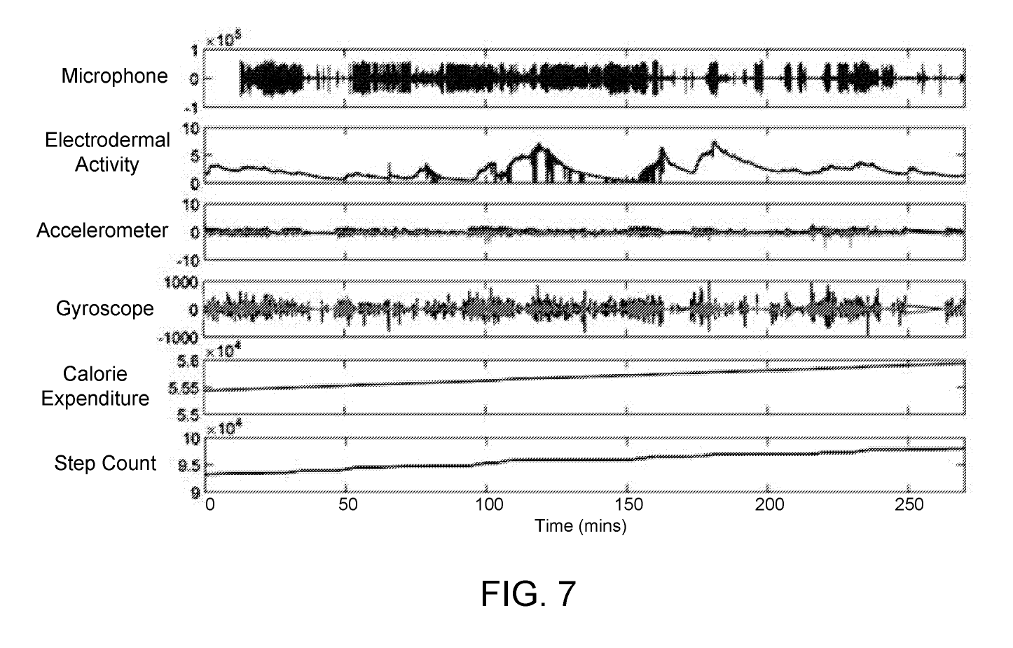

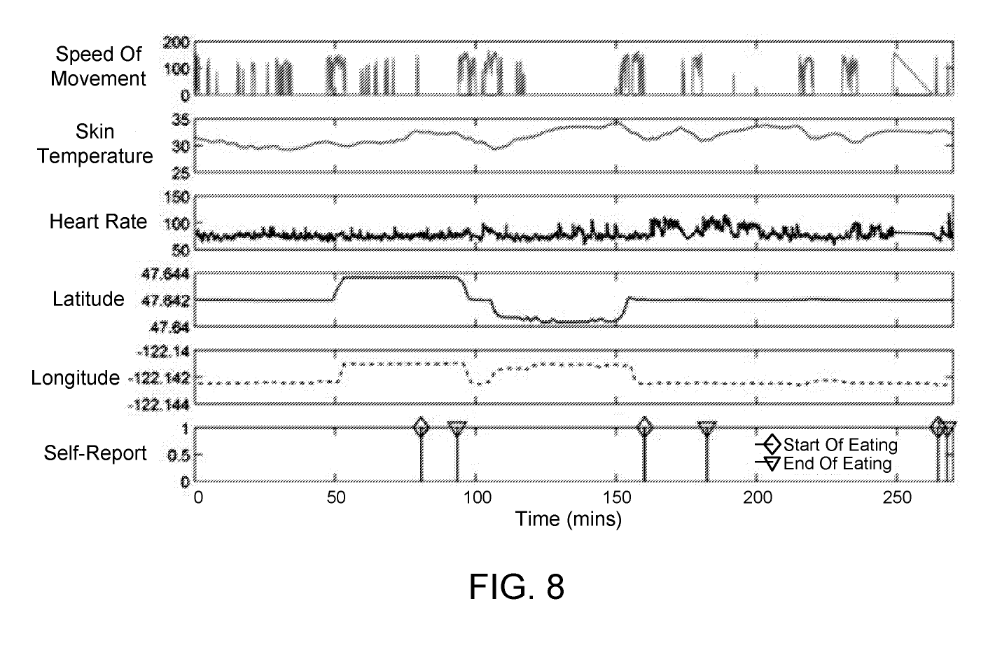

FIGS. 7 and 8 illustrate an exemplary set of time-stamped data streams, in simplified form, that is received from the set of mobile sensors. More particularly, FIG. 7 illustrates a time-stamped data stream labeled “Microphone” that includes current non-speech body sounds that are conducted through the body surface of a user. FIG. 7 also illustrates a time-stamped data stream labeled “Electrodermal Activity” that includes the current electrodermal activity of the user. FIG. 7 also illustrates a time-stamped data stream labeled “Accelerometer” that includes the current 3D linear velocity of the user. FIG. 7 also illustrates a time-stamped data stream labeled “Gyroscope” that includes the current 3D angular velocity of the user. FIG. 7 also illustrates a time-stamped data stream labeled “Calorie Expenditure” that includes a current cumulative value for the calorie expenditure of the user. FIG. 7 also illustrates a time-stamped data stream labeled “Step Count” that includes a current cumulative value for the step count of the user. FIG. 8 illustrates a time-stamped data stream labeled “Speed Of Movement” that includes the current speed of movement of an arm of the user. FIG. 8 also illustrates a time-stamped data stream labeled “Skin Temperature” that includes the current skin temperature of the user. FIG. 8 also illustrates a time-stamped data stream labeled “Heart Rate” that includes the current heart rate of the user. FIG. 8 also illustrates a time-stamped data stream labeled “Latitude” that includes the current latitude of the user. FIG. 8 also illustrates a time-stamped data stream labeled “Longitude” that includes the current longitude of the user. FIG. 8 also illustrates a time-stamped data stream labeled “Self Report” that includes information the user manually entered/logged into the aforementioned application that runs on the mobile computing device.

### 2.3 Feature Extraction

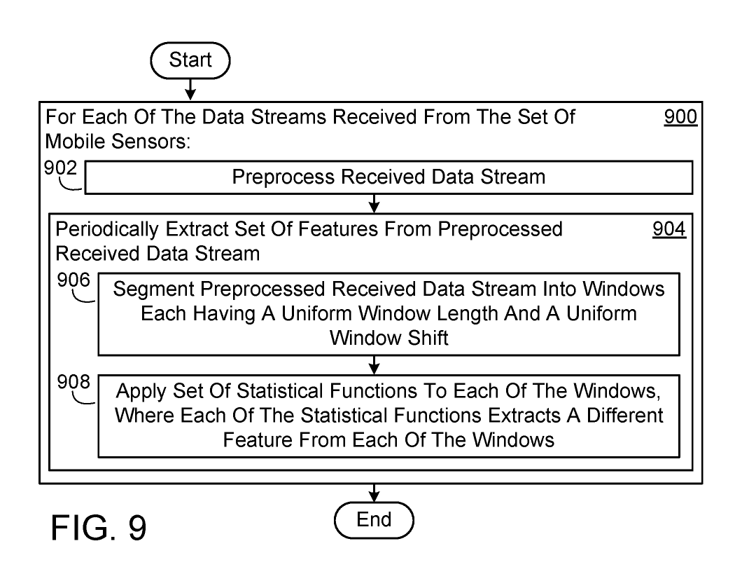

FIG. 9 illustrates an exemplary implementation, in simplified form, of a process for periodically extracting a set of features from the data stream that is received from each of the mobile sensors in the aforementioned set of mobile sensors. As exemplified in FIG. 9, the process starts with the following actions being performed for each of the data streams that is received from the set of mobile sensors (process action 900). First, the received data stream is preprocessed (process action 902). The particular type(s) of preprocessing that are performed on the received data stream depends on the particular type of mobile sensor that output the data stream and the particular type of physiological variable that is measured by this mobile sensor. By way of example but not limitation, whenever the data stream received from a given mobile sensor includes the current 3D linear velocity of a user, the received data stream preprocessing includes normalizing the received data stream. Whenever the data stream received from a given mobile sensor includes the current 3D angular velocity of a user, the received data stream preprocessing also includes normalizing the received data stream. Whenever the data stream received from a given mobile sensor includes a current cumulative value for the step count of a user, the received data stream preprocessing includes interpolating the received data stream and then using differentiation on the interpolated received data stream to estimate an instantaneous value for the step count of the user at each point in time. Whenever the data stream received from a given mobile sensor includes a current cumulative value for the calorie expenditure of a user, the received data stream preprocessing also includes interpolating the received data stream and then using differentiation on the interpolated received data stream to estimate an instantaneous value for the calorie expenditure of the user at each point in time.

Whenever the data stream received from a given mobile sensor includes the current electrodermal activity of a user, the received data stream preprocessing includes the following actions. First, the mean of the received data stream is computed and this mean is subtracted from the received data stream. The resulting data stream is then decomposed into two different components, namely a slow-varying (e.g., long-term response) tonic component, and a fast-varying (e.g., instantaneous response) phasic component. In an exemplary implementation of the wearable system described herein the tonic component of the user's electrodermal activity is estimated by applying a low-pass signal-filter with a cutoff frequency of 0.05 Hz to the received data stream. In a tested version of this implementation a conventional Butterworth-type low-pass signal-filter was used. Other implementations of the wearable system are also possible that use other cutoff frequencies for the low-pass signal-filter and other types of low-pass signal-filters. In an exemplary implementation of the wearable system the phasic component of the user's electrodermal activity is estimated by applying a band-pass signal-filter with cutoff frequencies at 0.05 Hz and 1.0 Hz to the received data stream. Other implementations of the wearable system are also possible that use other cutoff frequencies for the band-pass signal-filter.

Whenever the data stream received from a given mobile sensor includes current non-speech body sounds that are conducted through the body surface of a user, the received data stream preprocessing includes detecting each of the eating events in this data stream. In an exemplary implementation of the wearable system described herein this eating event detection is performed using a conventional BodyBeat mastication and swallowing sound detection method that detects characteristic eating sounds (such as mastication and swallowing, among others) in the received data stream. Whenever the data stream is received from the aforementioned health/fitness tracking device, the received data stream preprocessing can optionally also include re-sampling the received data stream using a fixed sampling frequency. This re-sampling is applicable in situations where the sampling rate of the health/fitness tracking device varies slightly over time, and is thus advantageous since it insures that each of the data streams which are received from the health/fitness tracking device have a sampling frequency that is substantially constant across all the data in the received data stream.

Referring again to FIG. 9, after the received data stream has been preprocessed (action 902) a set of features is periodically extracted from the preprocessed received data stream (process action 904). In an exemplary implementation of the wearable system described herein this periodic feature extraction is performed as follows. First, the preprocessed received data stream is segmented into windows each of which has a prescribed uniform window length (e.g., time duration) and a prescribed uniform window shift (process action 906). Generally speaking, the window length determines the quality of the features that are being extracted from the preprocessed received data stream (e.g., some window lengths result in lower quality features, while other window lengths result in higher quality features). In a tested version of this implementation the window shift was set to one minute, different window lengths between five minutes and 120 minutes were tested, and an optimal window length within this range was selected empirically based on the performance of the about-to-eat moment classifier and the regression-based time-to-next-eating-event predictor.

Referring again to FIG. 9, after the preprocessed received data stream has been segmented into windows (action 906) a set of statistical functions is applied to each of the windows, where each of the statistical functions extracts a different feature from each of the windows (process action 908). It is noted that many different types of features may be extracted from each of the windows in the segmented preprocessed data stream. In an exemplary implementation of the wearable system described herein the features that may be extracted can be categorized as follows. One category of extracted features captures the data extremes within each of the windows. For example, one of the statistical functions may determine the minimum data value within each of the windows. Another one of the statistical functions may determine the maximum data value within each of the windows. Another category of extracted features captures the data averages within each of the windows. For example, one of the statistical functions may determine the mean data value within each of the windows. Another one of the statistical functions may determine the root mean square data value within each of the windows. Another category of extracted features captures the data quartiles within each of the windows. For example, one of the statistical functions may determine the first quartile of the data within each of the windows. Another one of the statistical functions may determine the second quartile of the data within each of the windows. Yet another one of the statistical functions may determine the third quartile of the data within each of the windows. Another category of extracted features captures the data dispersion within each of the windows. For example, one of the statistical functions may determine the standard deviation of the data within each of the windows. Another one of the statistical functions may determine the interquartile range of the data within each of the windows. Another category of extracted features captures the data peaks within each of the windows. For example, one of the statistical functions may determine the total number of data peaks within each of the windows. Another one of the statistical functions may determine the mean distance between successive data peaks within each of the windows. Yet another one of the statistical functions may determine the mean amplitude of the data peaks within each of the windows. Another category of extracted features captures the rate of data change within each of the windows. For example, one of the statistical functions may determine the mean crossing rate of the data within each of the windows (e.g., the mean frequency at which the data within a given window crosses the mean data value within the window). Another category of extracted features captures the shape of the data within each of the windows. For example, one of the statistical functions may determine the linear regression slope of the data within each of the windows. Another category of extracted features captures time-related information within each of the windows. For example, one of the statistical functions may determine the time that has elapsed since the beginning of the user's day. In an exemplary implementation of the wearable system the beginning of the user's day is the particular time in a given day for the user that the wearable system starts receiving one or more data streams from the set of mobile sensors. Another one of the statistical functions may determine the time that has elapsed since the last eating event for the user. In one implementation of the wearable system the time of the last eating event for the user may be determined from the aforementioned information that the user manually enters/logs into the application that runs on their mobile computing device. In another implementation of the wearable system the time of the last eating event for the user may be determined from the data stream received from the body conduction microphone that is physically attached to the body of the user. Yet another one of the statistical functions may determine the number of previous eating events for the user since the beginning of the user's day.

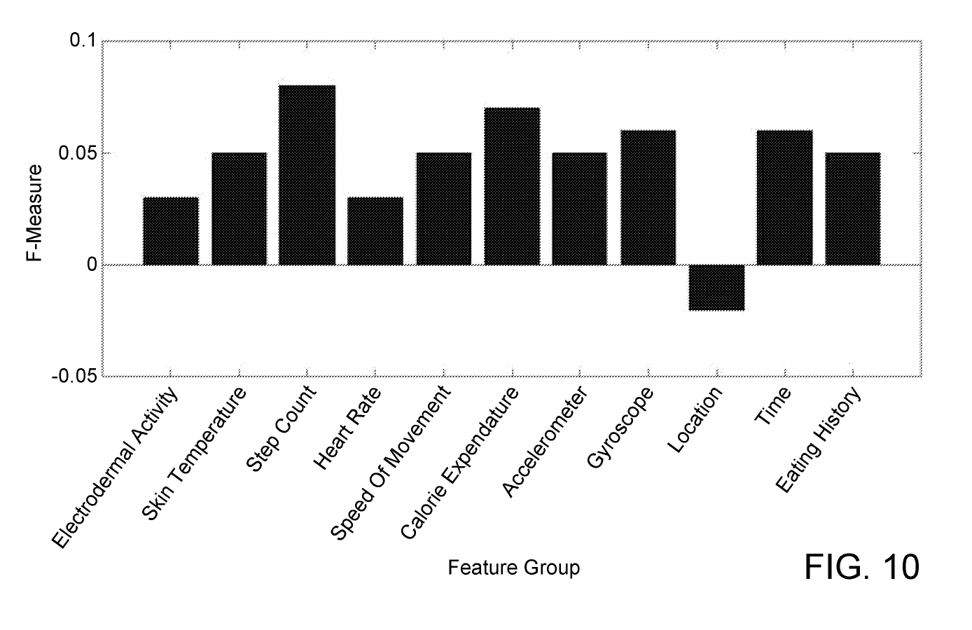

FIG. 10 illustrates the estimated contributions of different groups of features in the training of a user-independent about-to-eat moment classifier to predict about-to-eat moments for any user. More particularly, the contribution of each of the feature groups shown in FIG. 10 is estimated by measuring how much the performance of the classifier drops/decreases if the classifier is trained without the feature group. As exemplified in FIG. 10, the conventional F-measure (also known as the balanced F-score) metric was used to measure the performance of the classifier. As is shown in FIG. 10, none of the feature groups contribute a large drop/decrease in the performance of the classifier if they are not used to train the classifier. However, all of the feature groups except the location-related features (e.g., the latitude, longitude, and network location related features) contribute an increase in the performance of the classifier if they are used to train the classifier. It is interesting to note that the top contributing feature groups are the step-count-related features followed by the calorie-expenditure-related features. An intuitive basis for this might be that the step count of a user at a certain time (e.g., lunchtime) from a certain location (e.g., the user's home or workplace) toward another location such as a restaurant or cafe could be indicative of an about-to-eat moment for the user. Similarly, a certain calorie expenditure value for a user could be an indirect indicator of hunger or craving and thus could also be indicative of an about-to-eat moment for the user. It is also interesting to note that the gyroscope-related features contributed more than the accelerometer-related features. An intuitive basis for this might be that the gyroscope-related features may capture the characteristic hand gestures from user activities prior to an eating event such as typing on a keyboard, or opening a door, or walking, or the like. It is also interesting to note that the current time also contributed significantly which is intuitive since a user's eating is generally governed by a routine. It is also interesting to note that both the electrodermal-activity-related features and the heart-rate-related features contributed the least. If the classifier is trained without the location-related features the performance of the classifier increases; this is due to the fact that each user generally has a different location at a given point in time so the location-related features will generally be different for each user.

### 2.4 About-To-Eat Moment Classifier Training

Each of the windows of extracted features that lie within the boundary of the aforementioned about-to-eat definition window is labeled as an about-to-eat moment. Each of the windows of extracted features that lie outside the boundary of the about-to-eat definition window is labeled as a not-about-to-eat moment. The about-to-eat moment classifier is trained to distinguish between about-to-eat moments and not-about-to-eat moments using conventional machine learning methods. Since, as described heretofore, the location-related features introduced noise in the extracted feature space when these features are used to train a user-independent about-to-eat moment classifier, no location-related features were used to train the user-independent about-to-eat moment classifier.

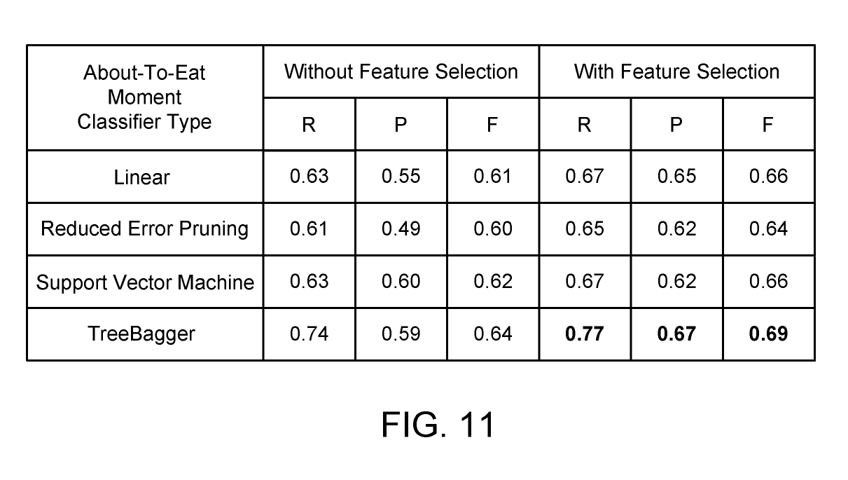

FIG. 11 illustrates the performance of the aforementioned different types of about-to-eat moment classifiers after they have been trained using the wearable system implementations described herein. As exemplified in FIG. 11, the performance of each of the different types of about-to-eat moment classifiers is measured in terms of recall (R), precision (P) and F-measure (F) using a conventional Leave-One-Person-Out (LOPO) cross-validation method and the conventional WEKA (Waikato Environment for Knowledge Analysis) suite of machine learning software. As is shown in FIG. 11, the TreeBagger type classifier exhibits the highest performance when the aforementioned selected subset of the features is used to train the TreeBagger type classifier.

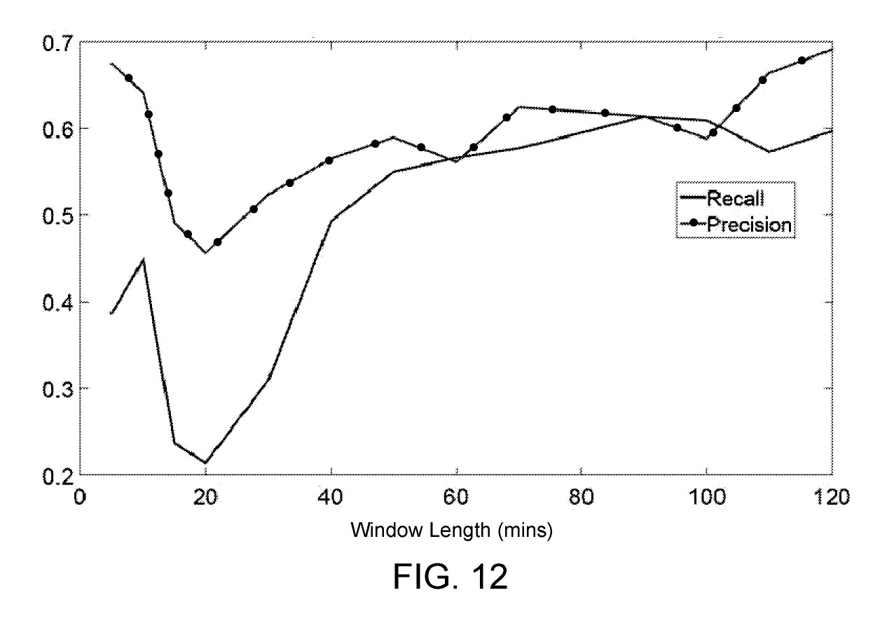

FIG. 12 illustrates how the performance of the TreeBagger type about-to-eat moment classifier changes as the aforementioned uniform window length that is used for the periodic feature extraction is incrementally changed from five minutes to 120 minutes. The performance measurement data shown in FIG. 12 was collected with the size of the aforementioned about-to-eat definition window being set to 30 minutes. As exemplified in FIG. 12, both very small and very large window lengths result in an increased performance of the classifier. In fact, the highest performance of the classifier is achieved (e.g., the highest quality features are extracted) when the window length is set to 120 minutes.

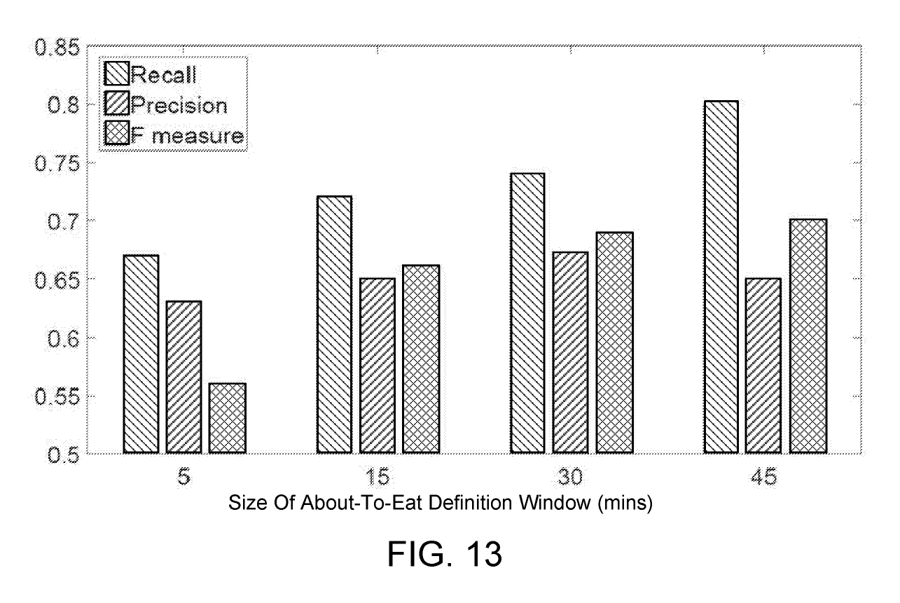

FIG. 13 illustrates how the performance of the TreeBagger type user-independent about-to-eat moment classifier changes as the size of the about-to-eat definition window is changed. Generally speaking, as the size of the about-to-eat definition window is increased the about-to-eat moments become more stringent. Changing the size of the about-to-eat definition window also affects the performance of the classifier. The following trade-off exists in selecting the size of the about-to-eat definition window. As exemplified in FIG. 13, as the size of the about-to-eat definition window is increased the performance of the classifier generally increases. This intuitively makes sense since increasing the size of the about-to-eat definition window gives the classifier more opportunity to capture subtle patterns in the extracted feature space and accurately predict the about-to-eat moments for a user. However, larger sizes of the about-to-eat definition window are less useful since, as described heretofore, an intervention is most effective when it occurs just before a person starts to perform an activity that the intervention is intended to prevent from happening or curtail (e.g., when the intervention occurs as close as possible to the beginning of a user's next eating event).

### 2.5 Regression-Based Time-To-Next-Eating-Event Predictor Training

The time remaining until the onset of the next eating event for a user is estimated from the endpoint of each of the windows that each of the aforementioned preprocessed received data streams is segmented into. If this time remaining from the endpoint of any particular window is greater than or equal to a prescribed time remaining threshold, the set of features that are extracted from this particular window is ignored (e.g., this set of features is not used to train the regression-based time-to-next-eating-event predictor) since this particular window is assumed to capture a non-eating life event (such as sleeping, among other types of non-eating life events). In tested version of the wearable system implementations described herein the time remaining threshold was set to five hours.

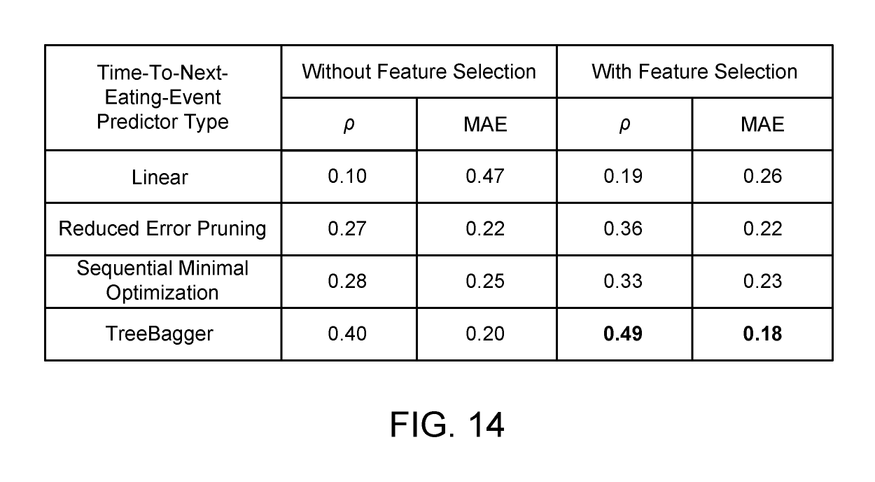

FIG. 14 illustrates the performance of the aforementioned different types of regression-based time-to-next-eating-event predictors after they have been trained using the wearable system implementations described herein. As exemplified in FIG. 14, the performance of each of the different types of regression-based time-to-next-eating-event predictors is measured in terms of the conventional Pearson correlation coefficient (ρ) and mean absolute error (MAE) using the aforementioned Leave-One-Person-Out (LOPO) cross-validation method and the conventional WEKA suite of machine learning software. As is shown in FIG. 14, the TreeBagger type predictor exhibits the highest performance when the aforementioned selected subset of the features is used to train the TreeBagger type predictor.

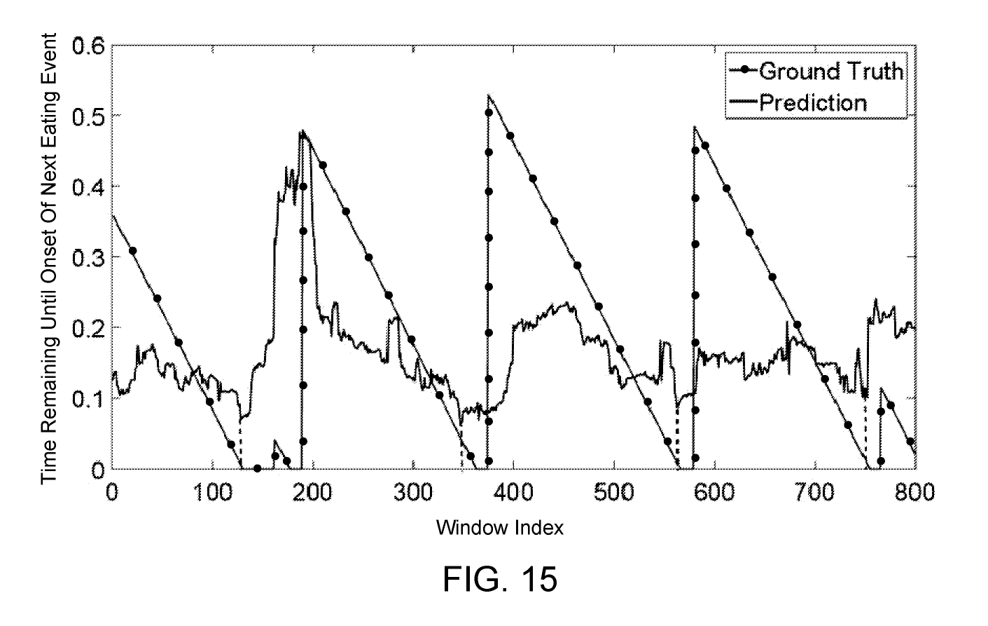

FIG. 15 illustrates how the time remaining until the onset of the next eating event for a user that is predicted by a TreeBagger type user-independent regression-based time-to-next-eating-event predictor performs with respect to a ground truth reference, where the predictor is trained using the selected subset of the features. The ground truth reference is considered to be zero during each eating event for the user. As exemplified in FIG. 15, the predictor exhibits the highest performance just before the start of an eating event.

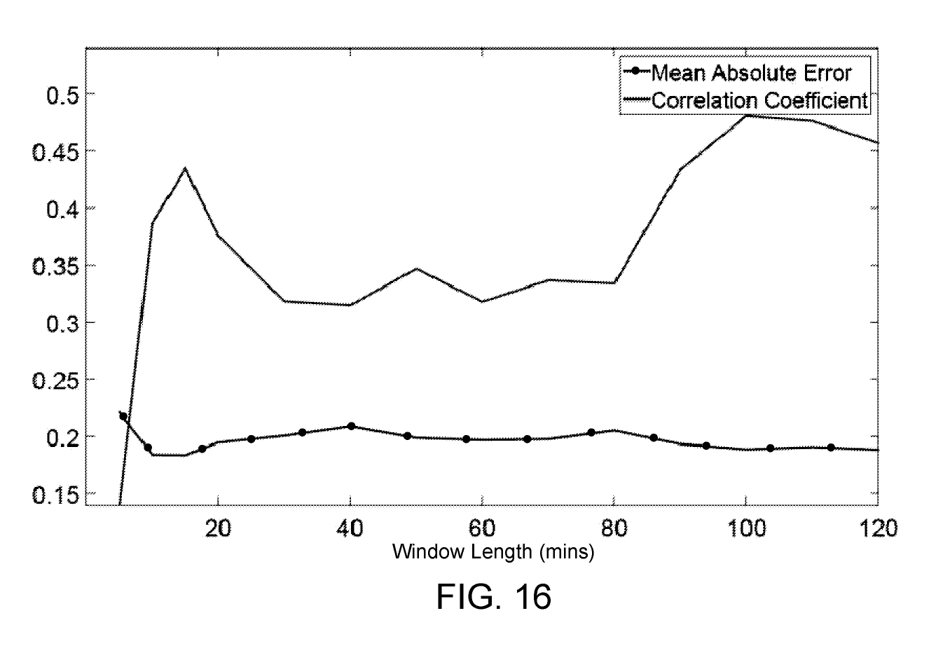

FIG. 16 illustrates how the performance of the TreeBagger type user-independent regression-based time-to-next-eating-event predictor changes as the aforementioned uniform window length that is used for periodic feature extraction is incrementally changed from five minutes to 120 minutes. As exemplified in FIG. 16, the highest performance of the predictor is achieved (e.g., the highest quality features are extracted) when the window length is set to 100 minutes. Features extracted with window lengths less than or greater than 100 minutes fail to capture the full dynamics of users' about-to-eat moments and thus result in a degradation of the predictor's performance.

### 3.0 Other Implementations

While the wearable system has been described by specific reference to implementations thereof, it is understood that variations and modifications thereof can be made without departing from the true spirit and scope of the wearable system. By way of example but not limitation, in addition to using the data streams that are received from the set of mobile sensors to train a machine-learned eating event predictor as described heretofore, an alternate implementation of the wearable system is possible where these data streams may be used to predict a user's craving and hunger during their about-to-eat moments. Additionally, the performance of the machine-learned eating event predictor may be further increased by selecting a set of user-specific features that incorporate the idiosyncrasies of a specific user (e.g., their specific eating pattern, lifestyle, and the like). For example, in a tested implementation of the wearable system described herein where a TreeBagger type user-dependent about-to-eat moment classifier was trained to predict about-to-eat moments for a specific user, the about-to-eat moment classifier exhibited a recall of 0.85, a precision of 0.82, and an F-measure of 0.84. Similarly, in another tested implementation of the wearable system where a TreeBagger type user-dependent regression-based time-to-next-eating-event predictor was trained to predict the time remaining until the onset of the next eating event for a specific user, the time-to-next-eating-event predictor exhibited a Pearson correlation coefficient of 0.65.

It is noted that any or all of the aforementioned implementations throughout the description may be used in any combination desired to form additional hybrid implementations. In addition, although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What has been described above includes example implementations. It is, of course, not possible to describe every conceivable combination of components or methodologies for purposes of describing the claimed subject matter, but one of ordinary skill in the art may recognize that many further combinations and permutations are possible. Accordingly, the claimed subject matter is intended to embrace all such alterations, modifications, and variations that fall within the spirit and scope of the appended claims.

In regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the foregoing implementations include a system as well as a computer-readable storage media having computer-executable instructions for performing the acts and/or events of the various methods of the claimed subject matter.

There are multiple ways of realizing the foregoing implementations (such as an appropriate application programming interface (API), tool kit, driver code, operating system, control, standalone or downloadable software object, or the like), which enable applications and services to use the implementations described herein. The claimed subject matter contemplates this use from the standpoint of an API (or other software object), as well as from the standpoint of a software or hardware object that operates according to the implementations set forth herein. Thus, various implementations described herein may have aspects that are wholly in hardware, or partly in hardware and partly in software, or wholly in software.

The aforementioned systems have been described with respect to interaction between several components. It will be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (e.g., hierarchical components).

Additionally, it is noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

### 4.0 Exemplary Operating Environments

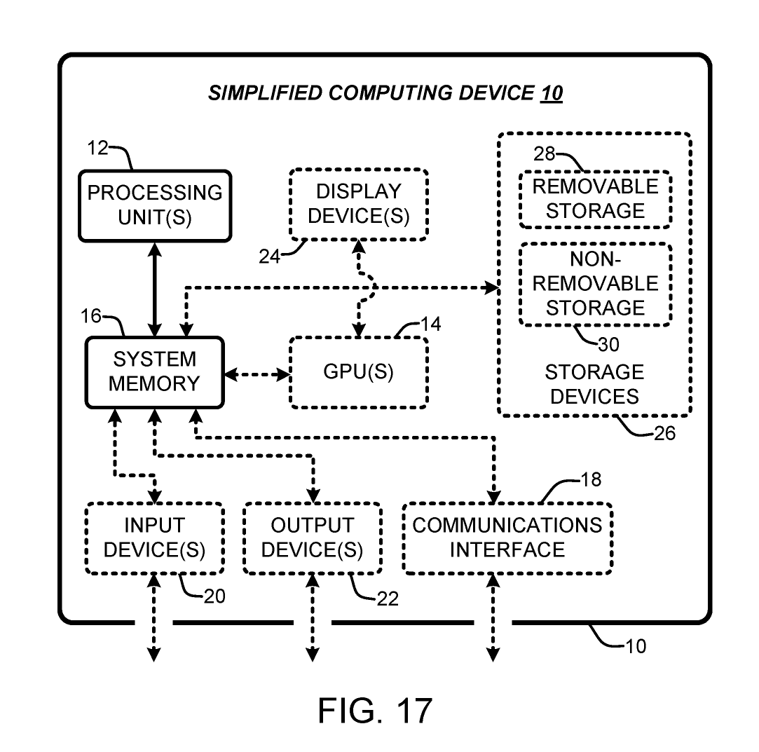

The wearable system implementations described herein are operational within numerous types of general purpose or special purpose computing system environments or configurations. FIG. 17 illustrates a simplified example of a general-purpose computer system on which various implementations and elements of the wearable system, as described herein, may be implemented. It is noted that any boxes that are represented by broken or dashed lines in the simplified computing device 10 shown in FIG. 17 represent alternate implementations of the simplified computing device. As described below, any or all of these alternate implementations may be used in combination with other alternate implementations that are described throughout this document. The simplified computing device 10 is typically found in devices having at least some minimum computational capability such as personal computers (PCs), server computers, handheld computing devices, laptop or mobile computers, communications devices such as cell phones and personal digital assistants (PDAs), multiprocessor systems, microprocessor-based systems, set top boxes, programmable consumer electronics, network PCs, minicomputers, mainframe computers, and audio or video media players.

To allow a device to realize the wearable system implementations described herein, the device should have a sufficient computational capability and system memory to enable basic computational operations. In particular, the computational capability of the simplified computing device 10 shown in FIG. 17 is generally illustrated by one or more processing unit(s) 12, and may also include one or more graphics processing units (GPUs) 14, either or both in communication with system memory 16. Note that that the processing unit(s) 12 of the simplified computing device 10 may be specialized microprocessors (such as a digital signal processor (DSP), a very long instruction word (VLIW) processor, a field-programmable gate array (FPGA), or other micro-controller) or can be conventional central processing units (CPUs) having one or more processing cores.

In addition, the simplified computing device 10 may also include other components, such as, for example, a communications interface 18. The simplified computing device 10 may also include one or more conventional computer input devices 20 (e.g., touchscreens, touch-sensitive surfaces, pointing devices, keyboards, audio input devices, voice or speech-based input and control devices, video input devices, haptic input devices, devices for receiving wired or wireless data transmissions, and the like) or any combination of such devices.

Similarly, various interactions with the simplified computing device 10 and with any other component or feature of the wearable system implementations described herein, including input, output, control, feedback, and response to one or more users or other devices or systems associated with the wearable system implementations, are enabled by a variety of Natural User Interface (NUI) scenarios. The NUI techniques and scenarios enabled by the wearable system implementations include, but are not limited to, interface technologies that allow one or more users user to interact with the wearable system implementations in a “natural” manner, free from artificial constraints imposed by input devices such as mice, keyboards, remote controls, and the like.

Such NUI implementations are enabled by the use of various techniques including, but not limited to, using NUI information derived from user speech or vocalizations captured via microphones or other sensors (e.g., speech and/or voice recognition). Such NUI implementations are also enabled by the use of various techniques including, but not limited to, information derived from a user's facial expressions and from the positions, motions, or orientations of a user's hands, fingers, wrists, arms, legs, body, head, eyes, and the like, where such information may be captured using various types of 2D or depth imaging devices such as stereoscopic or time-of-flight camera systems, infrared camera systems, RGB (red, green and blue) camera systems, and the like, or any combination of such devices. Further examples of such NUI implementations include, but are not limited to, NUI information derived from touch and stylus recognition, gesture recognition (both onscreen and adjacent to the screen or display surface), air or contact-based gestures, user touch (on various surfaces, objects or other users), hover-based inputs or actions, and the like. Such NUI implementations may also include, but are not limited, the use of various predictive machine intelligence processes that evaluate current or past user behaviors, inputs, actions, etc., either alone or in combination with other NUI information, to predict information such as user intentions, desires, and/or goals. Regardless of the type or source of the NUI-based information, such information may then be used to initiate, terminate, or otherwise control or interact with one or more inputs, outputs, actions, or functional features of the wearable system implementations described herein.

However, it should be understood that the aforementioned exemplary NUI scenarios may be further augmented by combining the use of artificial constraints or additional signals with any combination of NUI inputs. Such artificial constraints or additional signals may be imposed or generated by input devices such as mice, keyboards, and remote controls, or by a variety of remote or user worn devices such as accelerometers, electromyography (EMG) sensors for receiving myoelectric signals representative of electrical signals generated by user's muscles, heart-rate monitors, galvanic skin conduction sensors for measuring user perspiration, wearable or remote biosensors for measuring or otherwise sensing user brain activity or electric fields, wearable or remote biosensors for measuring user body temperature changes or differentials, and the like, or any of the other types of mobile sensors that have been described heretofore. Any such information derived from these types of artificial constraints or additional signals may be combined with any one or more NUI inputs to initiate, terminate, or otherwise control or interact with one or more inputs, outputs, actions, or functional features of the wearable system implementations described herein.

The simplified computing device 10 may also include other optional components such as one or more conventional computer output devices 22 (e.g., display device(s) 24, audio output devices, video output devices, devices for transmitting wired or wireless data transmissions, and the like). Note that typical communications interfaces 18, input devices 20, output devices 22, and storage devices 26 for general-purpose computers are well known to those skilled in the art, and will not be described in detail herein.

The simplified computing device 10 shown in FIG. 17 may also include a variety of computer-readable media. Computer-readable media can be any available media that can be accessed by the computer 10 via storage devices 26, and can include both volatile and nonvolatile media that is either removable 28 and/or non-removable 30, for storage of information such as computer-readable or computer-executable instructions, data structures, programs, sub-programs, or other data. Computer-readable media includes computer storage media and communication media. Computer storage media refers to tangible computer-readable or machine-readable media or storage devices such as digital versatile disks (DVDs), blu-ray discs (BD), compact discs (CDs), floppy disks, tape drives, hard drives, optical drives, solid state memory devices, random access memory (RAM), read-only memory (ROM), electrically erasable programmable read-only memory (EEPROM), CD-ROM or other optical disk storage, smart cards, flash memory (e.g., card, stick, and key drive), magnetic cassettes, magnetic tapes, magnetic disk storage, magnetic strips, or other magnetic storage devices. Further, a propagated signal is not included within the scope of computer-readable storage media.

Retention of information such as computer-readable or computer-executable instructions, data structures, programs, sub-programs, and the like, can also be accomplished by using any of a variety of the aforementioned communication media (as opposed to computer storage media) to encode one or more modulated data signals or carrier waves, or other transport mechanisms or communications protocols, and can include any wired or wireless information delivery mechanism. Note that the terms “modulated data signal” or “carrier wave” generally refer to a signal that has one or more of its characteristics set or changed in such a manner as to encode information in the signal. For example, communication media can include wired media such as a wired network or direct-wired connection carrying one or more modulated data signals, and wireless media such as acoustic, radio frequency (RF), infrared, laser, and other wireless media for transmitting and/or receiving one or more modulated data signals or carrier waves.

Furthermore, software, programs, sub-programs, and/or computer program products embodying some or all of the various wearable system implementations described herein, or portions thereof, may be stored, received, transmitted, or read from any desired combination of computer-readable or machine-readable media or storage devices and communication media in the form of computer-executable instructions or other data structures. Additionally, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, or media.

The wearable system implementations described herein may be further described in the general context of computer-executable instructions, such as programs and sub-programs, being executed by a computing device. Generally, sub-programs include routines, programs, objects, components, data structures, and the like, that perform particular tasks or implement particular abstract data types. The wearable system implementations may also be practiced in distributed computing environments where tasks are performed by one or more remote processing devices, or within a cloud of one or more devices, that are linked through one or more communications networks. In a distributed computing environment, sub-programs may be located in both local and remote computer storage media including media storage devices. Additionally, the aforementioned instructions may be implemented, in part or in whole, as hardware logic circuits, which may or may not include a processor.

Alternatively, or in addition, the functionality described herein can be performed, at least in part, by one or more hardware logic components. For example, and without limitation, illustrative types of hardware logic components that can be used include FPGAs, application-specific integrated circuits (ASICs), application-specific standard products (ASSPs), system-on-a-chip systems (SOCs), complex programmable logic devices (CPLDs), and so on.

### 5.0 Claim Support and Further Implementations

The following paragraphs summarize various examples of implementations which may be claimed in the present document. However, it should be understood that the implementations summarized below are not intended to limit the subject matter which may be claimed in view of the foregoing descriptions. Further, any or all of the implementations summarized below may be claimed in any desired combination with some or all of the implementations described throughout the foregoing description and any implementations illustrated in one or more of the figures, and any other implementations described below. In addition, it should be noted that the following implementations are intended to be understood in view of the foregoing description and figures described throughout this document.

In one implementation, a system is employed for predicting eating events for a user. This system includes a set of mobile sensors, each of the mobile sensors being configured to continuously measure a different physiological variable associated with the user and output a time-stamped data stream that includes the current value of this variable. The system also includes an eating event forecaster that includes one or more computing devices, these computing devices being in communication with each other via a computer network whenever there is a plurality of computing devices, and a computer program having a plurality of sub-programs executable by the one or more computing devices. The one or more computing devices are directed by the sub-programs of the computer program to, for each of the mobile sensors, receive the data stream output from the mobile sensor, and periodically extract a set of features from this received data stream, these features, which are among many features that can be extracted from this received data stream, having been determined to be specifically indicative of an about-to-eat moment, input the set of features that is periodically extracted from the data stream received from each of the mobile sensors into an about-to-eat moment classifier that has been trained to predict when the user is in an about-to-eat moment based on this set of features, and whenever an output of the classifier indicates that the user is currently in an about-to-eat moment, notify the user with a just-in-time eating intervention.

In one implementation of the just-described system, the mobile sensors include one or more of: a wearable computing device that is physically attached to the body of the user and provides health and fitness tracking functionality for the user; or a mobile computing device that is carried by the user. In another implementation the mobile sensors include one or more of: a heart rate sensor that is physically attached to the body of the user; or a skin temperature sensor that is physically attached to the body of the user; or an accelerometer that is physically attached to or carried by the user; or a gyroscope that is physically attached to or carried by the user; or a global positioning system sensor that is physically attached to or carried by the user; or an electrodermal activity sensor that is physically attached to the body of the user; or a body conduction microphone that is physically attached to the body of the user. In another implementation, the classifier includes one of: a linear type classifier; or a reduced error pruning type classifier; or a support vector machine type classifier; or a TreeBagger type classifier.

In another implementation one of the computing devices includes a mobile computing device that is carried by the user, and the user notification includes one or more of: a message that is displayed on a display screen of the mobile computing device; or an audible alert that is output from the mobile computing device; or a haptic alert that is output from the mobile computing device. In another implementation the received data stream includes one of: the current heart rate of the user; or the current skin temperature of the user; or the current three-dimensional linear velocity of the user; or the current three-dimensional angular velocity of the user; or the current longitude of the user; or the current latitude of the user; or the current electrodermal activity of the user; or current non-speech body sounds that are conducted through the body surface of the user, these sounds including the chewing and swallowing sounds of the user; or a current cumulative value for the step count of the user; or a current cumulative value for the calorie expenditure of the user; or the current speed of movement of an arm of the user.

In another implementation the sub-program for periodically extracting a set of features from the received data stream includes sub-programs for: preprocessing the received data stream; and periodically extracting the set of features from the preprocessed received data stream, this periodic extraction including sub-programs for, segmenting the preprocessed received data stream into windows each of which includes a prescribed uniform window length and a prescribed uniform window shift, and applying a set of statistical functions to each of these windows, each of the statistical functions extracting a different feature from each of these windows. In another implementation the sub-program for preprocessing the received data stream includes sub-programs for: whenever the received data stream includes the current three-dimensional linear velocity of the user, normalizing the received data stream; whenever the received data stream includes the current three-dimensional angular velocity of the user, normalizing the received data stream; whenever the received data stream includes a current cumulative value for the step count of the user, interpolating the received data stream, and using differentiation on the interpolated received data stream to estimate an instantaneous value for the step count of the user at each point in time; whenever the received data stream includes a current cumulative value for the calorie expenditure of the user, interpolating the received data stream, and using differentiation on the interpolated received data stream to estimate an instantaneous value for the calorie expenditure of the user at each point in time; whenever the received data stream includes the current electrodermal activity of the user, computing the mean of the received data stream, subtracting this mean from the received data stream, and decomposing the resulting data stream into a slow-varying tonic component and a fast-varying phasic component; and whenever the received data stream includes current non-speech body sounds that are conducted through the body surface of the user, detecting each of the eating events in the received data stream. In another implementation, the sub-program for detecting each of the eating events in the received data stream includes a sub-program for using a BodyBeat mastication and swallowing sound detection method to detect characteristic eating sounds in the received data stream.

In another implementation the set of features that is periodically extracted from the preprocessed received data stream includes two or more of: the minimum data value within each of the windows; or the maximum data value within each of the windows; or the mean data value within each of the windows; or the root mean square data value within each of the windows; or the first quartile of the data within each of the windows; or the second quartile of the data within each of the windows; or the third quartile of the data within each of the windows; or the standard deviation of the data within each of the windows; or the interquartile range of the data within each of the windows; or the total number of data peaks within each of the windows; or the mean distance between successive data peaks within each of the windows; or the mean amplitude of the data peaks within each of the windows; or the mean crossing rate of the data within each of the windows; or the linear regression slope of the data within each of the windows; or the time that has elapsed since the beginning of the day for the user; or the time that has elapsed since the last eating event for the user; or the number of previous eating events for the user since the beginning of the day for the user.

The implementations described in any of the previous paragraphs in this section may also be combined with each other, and with one or more of the implementations and versions described prior to this section. For example, some or all of the preceding implementations and versions may be combined with the foregoing implementation where the classifier includes one of: a linear type classifier; or a reduced error pruning type classifier; or a support vector machine type classifier; or a TreeBagger type classifier. In addition, some or all of the preceding implementations may be combined with the foregoing implementation where the sub-program for periodically extracting a set of features from the received data stream includes sub-programs for: preprocessing the received data stream; and periodically extracting the set of features from the preprocessed received data stream, this periodic extraction including sub-programs for, segmenting the preprocessed received data stream into windows each of which includes a prescribed uniform window length and a prescribed uniform window shift, and applying a set of statistical functions to each of these windows, each of the statistical functions extracting a different feature from each of these windows.

In another implementation, a system is employed for predicting eating events for a user. This system includes a set of mobile sensors, each of the mobile sensors being configured to continuously measure a different physiological variable associated with the user and output a time-stamped data stream that includes the current value of this variable. The system also includes an eating event forecaster that includes one or more computing devices, these computing devices being in communication with each other via a computer network whenever there is a plurality of computing devices, and a computer program having a plurality of sub-programs executable by the one or more computing devices, the one or more computing devices being directed by the sub-programs of the computer program to, for each of the mobile sensors, receive the data stream output from the mobile sensor, and periodically extract a set of features from this received data stream, these features, which are among many features that can be extracted from this received data stream, having been determined to be specifically indicative of an about-to-eat moment, input the set of features that is periodically extracted from the data stream received from each of the mobile sensors into a regression-based time-to-next-eating-event predictor that has been trained to predict the time remaining until the onset of the next eating event for the user based on this set of features, and whenever an output of the predictor indicates that the current time remaining until the onset of the next eating event for the user is less than a prescribed threshold, notify the user with a just-in-time eating intervention.

In one implementation of the just-described system, the predictor includes one of: a linear type predictor; or a reduced error pruning type predictor; or a sequential minimal optimization type predictor; or a TreeBagger type predictor. In another implementation one of the computing devices includes a mobile computing device that is carried by the user, and the user notification includes one or more of: a message that is displayed on a display screen of the mobile computing device; or an audible alert that is output from the mobile computing device; or a haptic alert that is output from the mobile computing device. In another implementation the received data stream includes one of: the current heart rate of the user; or the current skin temperature of the user; or the current three-dimensional linear velocity of the user; or the current three-dimensional angular velocity of the user; or the current longitude of the user; or the current latitude of the user; or the current electrodermal activity of the user; or current non-speech body sounds that are conducted through the body surface of the user, these sounds including the chewing and swallowing sounds of the user; or a current cumulative value for the step count of the user; or a current cumulative value for the calorie expenditure of the user; or the current speed of movement of an arm of the user.

In another implementation the sub-program for periodically extracting a set of features from the received data stream includes sub-programs for: preprocessing the received data stream; and periodically extracting the set of features from the preprocessed received data stream, this periodic extraction including sub-programs for, segmenting the preprocessed received data stream into windows each of which includes a prescribed uniform window length and a prescribed uniform window shift, and applying a set of statistical functions to each of these windows, each of the statistical functions extracting a different feature from each of these windows. In another implementation the set of features that is periodically extracted from the preprocessed received data stream includes two or more of: the minimum data value within each of the windows; or the maximum data value within each of the windows; or the mean data value within each of the windows; or the root mean square data value within each of the windows; or the first quartile of the data within each of the windows; or the second quartile of the data within each of the windows; or the third quartile of the data within each of the windows; or the standard deviation of the data within each of the windows; or the interquartile range of the data within each of the windows; or the total number of data peaks within each of the windows; or the mean distance between successive data peaks within each of the windows; or the mean amplitude of the data peaks within each of the windows; or the mean crossing rate of the data within each of the windows; or the linear regression slope of the data within each of the windows; or the time that has elapsed since the beginning of the day for the user; or the time that has elapsed since the last eating event for the user; or the number of previous eating events for the user since the beginning of the day for the user.

As indicated previously, the implementations described in any of the previous paragraphs in this section may also be combined with each other, and with one or more of the implementations and versions described prior to this section. For example, some or all of the preceding implementations and versions may be combined with the foregoing implementation where the sub-program for periodically extracting a set of features from the received data stream includes sub-programs for: preprocessing the received data stream; and periodically extracting the set of features from the preprocessed received data stream, this periodic extraction including sub-programs for, segmenting the preprocessed received data stream into windows each of which includes a prescribed uniform window length and a prescribed uniform window shift, and applying a set of statistical functions to each of these windows, each of the statistical functions extracting a different feature from each of these windows.

In another implementation, a system is employed for training a machine-learned eating event predictor. This system includes a set of mobile sensors, each of the mobile sensors being configured to continuously measure a different physiological variable associated with each of one or more users and output a time-stamped data stream that includes the current value of this variable. The system also includes an eating event prediction trainer that includes one or more computing devices, these computing devices being in communication with each other via a computer network whenever there is a plurality of computing devices, and a computer program having a plurality of sub-programs executable by the one or more computing devices, the one or more computing devices being directed by the sub-programs of the computer program to, for each of the mobile sensors, receive the data stream output from the mobile sensor, and periodically extract a set of features from this received data stream, these features, which are among many features that can be extracted from this received data stream, having been determined to be specifically indicative of an about-to-eat moment, use the set of features that is periodically extracted from the data stream received from each of the mobile sensors to train the predictor to predict when an eating event for a user is about to occur, and output the trained predictor.

In one implementation of the just-described system, the predictor includes an about-to-eat moment classifier that is trained to predict when a user is in an about-to-eat moment. In another implementation the predictor includes a regression-based time-to-next-eating-event predictor, the sub-program for periodically extracting a set of features from the received data stream includes a sub-program for mapping each of the features in the set of features that is periodically extracted from the received data stream to the current time remaining until the next eating event, this current time remaining being determined by analyzing the data stream received from each of the mobile sensors, and the sub-program for using the set of features that is periodically extracted from the data stream received from each of the mobile sensors to train the predictor to predict when an eating event for a user is about to occur includes a sub-program for using the set of features that is periodically extracted from the data stream received from each of the mobile sensors in combination with this mapping of each of the features in this set of features to train the time-to-next-eating-event predictor to predict the time remaining until the onset of the next eating event for the user.

In another implementation the sub-program for using the set of features that is periodically extracted from the data stream received from each of the mobile sensors to train the predictor to predict when an eating event for a user is about to occur includes sub-programs for: inputting the set of features that is periodically extracted from the data stream received from each of the mobile sensors into an overall set of features; using a combination of a correlation-based feature selection method and a best-first decision tree machine learning method to select a subset of the features in the overall set of features; and using the selected subset of the features to train the predictor to predict when an eating event for a user is about to occur.

In one implementation, an eating event prediction system is implemented by a means for predicting eating events for a user. The eating event prediction system includes a set of mobile sensing means for continuously measuring physiological variables associated with the user, each of the mobile sensing means being configured to continuously measure a different physiological variable associated with the user and output a time-stamped data stream that includes the current value of this variable. The eating event prediction system also includes a forecasting means for forecasting eating events that includes one or more computing devices, these computing devices being in communication with each other via a computer network whenever there is a plurality of computing devices, these computing devices including processors configured to execute, for each of the mobile sensing means, a data reception step for receiving the data stream output from the mobile sensing means, and a feature extraction step for periodically extracting a set of features from this received data stream, these features, which are among many features that can be extracted from this received data stream, having been determined to be specifically indicative of an about-to-eat moment, an inputting step for inputting the set of features that is periodically extracted from the data stream received from each of the mobile sensing means into a classification means for predicting about-to-eat moments that has been trained to predict when the user is in an about-to-eat moment based on this set of features, and whenever an output of the classification means indicates that the user is currently in an about-to-eat moment, a user notification step for notifying the user with a just-in-time eating intervention.

In one implementation of the just-described eating event prediction system the mobile sensing means include one or more of: a wearable computing device that is physically attached to the body of the user and provides health and fitness tracking functionality for the user; or a mobile computing device that is carried by the user. In another implementation the mobile sensing means includes one or more of: a heart rate sensor that is physically attached to the body of the user; or a skin temperature sensor that is physically attached to the body of the user; or an accelerometer that is physically attached to or carried by the user; or a gyroscope that is physically attached to or carried by the user; or a global positioning system sensor that is physically attached to or carried by the user; or an electrodermal activity sensor that is physically attached to the body of the user; or a body conduction microphone that is physically attached to the body of the user. In another implementation the classification means includes one of: a linear type classifier; or a reduced error pruning type classifier; or a support vector machine type classifier; or a TreeBagger type classifier.

In another implementation the feature extraction step for periodically extracting a set of features from the received data stream includes: a preprocessing step for preprocessing the received data stream; and a periodic extraction step for periodically extracting the set of features from the preprocessed received data stream, this periodic extraction step including, a segmentation step for segmenting the preprocessed received data stream into windows each of which includes a prescribed uniform window length and a prescribed uniform window shift, and a function application step for applying a set of statistical functions to each of these windows, each of the statistical functions extracting a different feature from each of these windows. In another implementation the preprocessing step for preprocessing the received data stream includes: whenever the received data stream includes the current three-dimensional linear velocity of the user, a normalization step for normalizing the received data stream; whenever the received data stream includes the current three-dimensional angular velocity of the user, a normalization step for normalizing the received data stream; whenever the received data stream includes a current cumulative value for the step count of the user, an interpolation step for interpolating the received data stream, and a differentiation step for using differentiation on the interpolated received data stream to estimate an instantaneous value for the step count of the user at each point in time; whenever the received data stream includes a current cumulative value for the calorie expenditure of the user, an interpolation step for interpolating the received data stream, and a differentiation step for using differentiation on the interpolated received data stream to estimate an instantaneous value for the calorie expenditure of the user at each point in time; whenever the received data stream includes the current electrodermal activity of the user, a mean computation step for computing the mean of the received data stream, a mean subtraction step for subtracting this mean from the received data stream, and a decomposition step for decomposing the resulting data stream into a slow-varying tonic component and a fast-varying phasic component; and whenever the received data stream includes current non-speech body sounds that are conducted through the body surface of the user, a detection step for detecting each of the eating events in the received data stream.

In one implementation, an eating event prediction system is implemented by a means for predicting eating events for a user. The eating event prediction system includes a set of mobile sensing means for continuously measuring physiological variables associated with the user, each of the mobile sensing means being configured to continuously measure a different physiological variable associated with the user and output a time-stamped data stream that includes the current value of this variable. The eating event prediction system also includes a forecasting means for forecasting eating events that includes one or more computing devices, these computing devices being in communication with each other via a computer network whenever there is a plurality of computing devices, these computing devices including processors configured to execute, for each of the mobile sensing means, a data reception step for receiving the data stream output from the mobile sensing means, and a feature extraction step for periodically extracting a set of features from this received data stream, these features, which are among many features that can be extracted from this received data stream, having been determined to be specifically indicative of an about-to-eat moment, an inputting step for inputting the set of features that is periodically extracted from the data stream received from each of the mobile sensing means into a regression-based prediction means for predicting the time remaining until the onset of an eating event that has been trained to predict the time remaining until the onset of the next eating event for the user based on this set of features, and whenever an output of the prediction means indicates that the current time remaining until the onset of the next eating event for the user is less than a prescribed threshold, a user notification step for notifying the user with a just-in-time eating intervention.

In one implementation of the just-described eating event prediction system the prediction means includes one of: a linear type predictor; or a reduced error pruning type predictor; or a sequential minimal optimization type predictor; or a TreeBagger type predictor. In another implementation the feature extraction step for periodically extracting a set of features from the received data stream includes: a preprocessing step for preprocessing the received data stream; and a periodic extraction step for periodically extracting the set of features from the preprocessed received data stream, this periodic extraction step including, a segmentation step for segmenting the preprocessed received data stream into windows each of which includes a prescribed uniform window length and a prescribed uniform window shift, and a function application step for applying a set of statistical functions to each of these windows, each of the statistical functions extracting a different feature from each of these windows.

In one implementation, a predictor training system is implemented by a means for training a machine-learned eating event predictor. The predictor training system includes a set of mobile sensing means for continuously measuring physiological variables associated with one or more users, each of the mobile sensing means being configured to continuously measure a different physiological variable associated with each of the one or more users and output a time-stamped data stream that includes the current value of this variable. The predictor training system also includes a training means for training the predictor that includes one or more computing devices, these computing devices being in communication with each other via a computer network whenever there is a plurality of computing devices, these computing devices including processors configured to execute, for each of the mobile sensing means, a data reception step for receiving the data stream output from the mobile sensing means, and a feature extraction step for periodically extracting a set of features from this received data stream, these features, which are among many features that can be extracted from this received data stream, having been determined to be specifically indicative of an about-to-eat moment, a feature utilization step for using the set of features that is periodically extracted from the data stream received from each of the mobile sensing means to train the predictor to predict when an eating event for a user is about to occur, and an outputting step for outputting the trained predictor.

In one implementation of the just-described predictor training system the predictor includes a regression-based time-to-next-eating-event predictor, the feature extraction step for periodically extracting a set of features from the received data stream includes a mapping step for mapping each of the features in the set of features that is periodically extracted from the received data stream to the current time remaining until the next eating event, this current time remaining being determined by analyzing the data stream received from each of the mobile sensing means, and the feature utilization step for using the set of features that is periodically extracted from the data stream received from each of the mobile sensing means to train the predictor to predict when an eating event for a user is about to occur includes a training step for using the set of features that is periodically extracted from the data stream received from each of the mobile sensing means in combination with the mapping of each of the features in this set of features to train the time-to-next-eating-event predictor to predict the time remaining until the onset of the next eating event for the user.

In another implementation the feature utilization step for using the set of features that is periodically extracted from the data stream received from each of the mobile sensing means to train the predictor to predict when an eating event for a user is about to occur includes: an inputting step for inputting the set of features that is periodically extracted from the data stream received from each of the mobile sensors into an overall set of features; a feature selection step for using a combination of a correlation-based feature selection method and a best-first decision tree machine learning method to select a subset of the features in the overall set of features; and a training step for using the selected subset of the features to train the predictor to predict when an eating event for a user is about to occur.

Wherefore, what is claimed is:

1. A system for predicting eating events for a user, comprising:
a set of mobile sensors, each of the mobile sensors being configured to continuously measure a different physiological variable associated with the user and output a time-stamped data stream comprising the current value of said variable; and
an eating event forecaster comprising one or more computing devices, said computing devices being in communication with each other via a computer network whenever there is a plurality of computing devices, and a computer program having a plurality of sub-programs executable by the one or more computing devices, the one or more computing devices being directed by the sub-programs of the computer program to,
for each of the mobile sensors,
receive the data stream output from the mobile sensor, and
periodically extract a set of features from said received data stream, said features, which are among many features that can be extracted from said received data stream, having been determined to be specifically indicative of an about-to-eat moment,
input the set of features that is periodically extracted from the data stream received from each of the mobile sensors into an about-to-eat moment classifier that has been trained to predict when the user is in an about-to-eat moment based on said set of features, and
whenever an output of the classifier indicates that the user is currently in an about-to-eat moment, notify the user with a just-in-time eating intervention.

2. The system of claim 1, wherein the mobile sensors comprise one or more of:
a wearable computing device that is physically attached to the body of the user and provides health and fitness tracking functionality for the user; or
a mobile computing device that is carried by the user.

3. The system of claim 1, wherein the mobile sensors comprise one or more of:
a heart rate sensor that is physically attached to the body of the user; or
a skin temperature sensor that is physically attached to the body of the user; or
an accelerometer that is physically attached to or carried by the user; or
a gyroscope that is physically attached to or carried by the user; or
a global positioning system sensor that is physically attached to or carried by the user; or
an electrodermal activity sensor that is physically attached to the body of the user; or
a body conduction microphone that is physically attached to the body of the user.

4. The system of claim 1, wherein the classifier comprises one of:
a linear type classifier; or
a reduced error pruning type classifier; or
a support vector machine type classifier; or
a TreeBagger type classifier.

5. The system of claim 1, wherein,
one of the computing devices comprises a mobile computing device that is carried by the user, and
said user notification comprises one or more of:
a message that is displayed on a display screen of the mobile computing device; or
an audible alert that is output from the mobile computing device; or
a haptic alert that is output from the mobile computing device.

6. The system of claim 1, wherein said received data stream comprises one of:
the current heart rate of the user; or
the current skin temperature of the user; or
the current three-dimensional linear velocity of the user; or
the current three-dimensional angular velocity of the user; or
the current longitude of the user; or
the current latitude of the user; or
the current electrodermal activity of the user; or
current non-speech body sounds that are conducted through the body surface of the user, said sounds comprising the chewing and swallowing sounds of the user; or
a current cumulative value for the step count of the user; or
a current cumulative value for the calorie expenditure of the user; or
the current speed of movement of an arm of the user.

7. The system of claim 1, wherein the sub-program for periodically extracting a set of features from said received data stream comprises sub-programs for:
preprocessing said received data stream; and
periodically extracting the set of features from the preprocessed received data stream, said periodic extraction comprising sub-programs for,
segmenting the preprocessed received data stream into windows each of which comprises a prescribed uniform window length and a prescribed uniform window shift, and
applying a set of statistical functions to each of said windows, each of the statistical functions extracting a different feature from each of said windows.

8. The system of claim 7, wherein the sub-program for preprocessing said received data stream comprises sub-programs for:
whenever said received data stream comprises the current three-dimensional linear velocity of the user, normalizing said received data stream;
whenever said received data stream comprises the current three-dimensional angular velocity of the user, normalizing said received data stream;
whenever said received data stream comprises a current cumulative value for the step count of the user, interpolating said received data stream, and using differentiation on the interpolated received data stream to estimate an instantaneous value for the step count of the user at each point in time;
whenever said received data stream comprises a current cumulative value for the calorie expenditure of the user, interpolating said received data stream, and using differentiation on the interpolated received data stream to estimate an instantaneous value for the calorie expenditure of the user at each point in time;
whenever said received data stream comprises the current electrodermal activity of the user, computing the mean of said received data stream, subtracting said mean from said received data stream, and decomposing the resulting data stream into a slow-varying tonic component and a fast-varying phasic component; and
whenever said received data stream comprises current non-speech body sounds that are conducted through the body surface of the user, detecting each of the eating events in said received data stream.

9. The system of claim 8, wherein the sub-program for detecting each of the eating events in said received data stream comprises a sub-program for using a BodyBeat mastication and swallowing sound detection method to detect characteristic eating sounds in said received data stream.

10. The system of claim 7, wherein the set of features that is periodically extracted from the preprocessed received data stream comprises two or more of:
the minimum data value within each of said windows; or
the maximum data value within each of said windows; or
the mean data value within each of said windows; or
the root mean square data value within each of said windows; or
the first quartile of the data within each of said windows; or
the second quartile of the data within each of said windows; or
the third quartile of the data within each of said windows; or
the standard deviation of the data within each of said windows; or
the interquartile range of the data within each of said windows; or
the total number of data peaks within each of said windows; or
the mean distance between successive data peaks within each of said windows; or
the mean amplitude of the data peaks within each of said windows; or
the mean crossing rate of the data within each of said windows; or
the linear regression slope of the data within each of said windows; or
the time that has elapsed since the beginning of the day for the user; or
the time that has elapsed since the last eating event for the user; or
the number of previous eating events for the user since the beginning of the day for the user.

11. A system for predicting eating events for a user, comprising:
a set of mobile sensors, each of the mobile sensors being configured to continuously measure a different physiological variable associated with the user and output a time-stamped data stream comprising the current value of said variable; and
an eating event forecaster comprising one or more computing devices, said computing devices being in communication with each other via a computer network whenever there is a plurality of computing devices, and a computer program having a plurality of sub-programs executable by the one or more computing devices, the one or more computing devices being directed by the sub-programs of the computer program to,
for each of the mobile sensors,
receive the data stream output from the mobile sensor, and
periodically extract a set of features from said received data stream, said features, which are among many features that can be extracted from said received data stream, having been determined to be specifically indicative of an about-to-eat moment,
input the set of features that is periodically extracted from the data stream received from each of the mobile sensors into a regression-based time-to-next-eating-event predictor that has been trained to predict the time remaining until the onset of the next eating event for the user based on said set of features, and
whenever an output of the predictor indicates that the current time remaining until the onset of the next eating event for the user is less than a prescribed threshold, notify the user with a just-in-time eating intervention.

12. The system of claim 11, wherein the predictor comprises one of:
a linear type predictor; or
a reduced error pruning type predictor; or
a sequential minimal optimization type predictor; or
a TreeBagger type predictor.

13. The system of claim 11, wherein,
one of the computing devices comprises a mobile computing device that is carried by the user, and
said user notification comprises one or more of:
a message that is displayed on a display screen of the mobile computing device; or
an audible alert that is output from the mobile computing device; or
a haptic alert that is output from the mobile computing device.

14. The system of claim 11, wherein said received data stream comprises one of:
the current heart rate of the user; or
the current skin temperature of the user; or
the current three-dimensional linear velocity of the user; or
the current three-dimensional angular velocity of the user; or
the current longitude of the user; or
the current latitude of the user; or
the current electrodermal activity of the user; or
current non-speech body sounds that are conducted through the body surface of the user, said sounds comprising the chewing and swallowing sounds of the user; or
a current cumulative value for the step count of the user; or
a current cumulative value for the calorie expenditure of the user; or
the current speed of movement of an arm of the user.

15. The system of claim 11, wherein the sub-program for periodically extracting a set of features from said received data stream comprises sub-programs for:
preprocessing said received data stream; and
periodically extracting the set of features from the preprocessed received data stream, said periodic extraction comprising sub-programs for,
segmenting the preprocessed received data stream into windows each of which comprises a prescribed uniform window length and a prescribed uniform window shift, and
applying a set of statistical functions to each of said windows, each of the statistical functions extracting a different feature from each of said windows.

16. The system of claim 15, wherein the set of features that is periodically extracted from the preprocessed received data stream comprises two or more of:
the minimum data value within each of said windows; or
the maximum data value within each of said windows; or
the mean data value within each of said windows; or
the root mean square data value within each of said windows; or
the first quartile of the data within each of said windows; or
the second quartile of the data within each of said windows; or
the third quartile of the data within each of said windows; or
the standard deviation of the data within each of said windows; or
the interquartile range of the data within each of said windows; or
the total number of data peaks within each of said windows; or
the mean distance between successive data peaks within each of said windows; or
the mean amplitude of the data peaks within each of said windows; or
the mean crossing rate of the data within each of said windows; or
the linear regression slope of the data within each of said windows; or
the time that has elapsed since the beginning of the day for the user; or
the time that has elapsed since the last eating event for the user; or
the number of previous eating events for the user since the beginning of the day for the user.

17. A system for training a machine-learned eating event predictor, comprising:
a set of mobile sensors, each of the mobile sensors being configured to continuously measure a different physiological variable associated with each of one or more users and output a time-stamped data stream comprising the current value of said variable; and
an eating event prediction trainer comprising one or more computing devices, said computing devices being in communication with each other via a computer network whenever there is a plurality of computing devices, and a computer program having a plurality of sub-programs executable by the one or more computing devices, the one or more computing devices being directed by the sub-programs of the computer program to,
for each of the mobile sensors,
receive the data stream output from the mobile sensor, and
periodically extract a set of features from said received data stream, said features, which are among many features that can be extracted from said received data stream, having been determined to be specifically indicative of an about-to-eat moment,
use the set of features that is periodically extracted from the data stream received from each of the mobile sensors to train the predictor to predict when an eating event for a user is about to occur, and
output the trained predictor.

18. The system of claim 17, wherein the predictor comprises an about-to-eat moment classifier that is trained to predict when a user is in an about-to-eat moment.

19. The system of claim 17, wherein,
the predictor comprises a regression-based time-to-next-eating-event predictor,
the sub-program for periodically extracting a set of features from said received data stream comprises a sub-program for mapping each of the features in the set of features that is periodically extracted from said received data stream to the current time remaining until the next eating event, said current time remaining being determined by analyzing the data stream received from each of the mobile sensors, and
the sub-program for using the set of features that is periodically extracted from the data stream received from each of the mobile sensors to train the predictor to predict when an eating event for a user is about to occur comprises a sub-program for using the set of features that is periodically extracted from the data stream received from each of the mobile sensors in combination with said mapping of each of the features in said set of features to train the time-to-next-eating-event predictor to predict the time remaining until the onset of the next eating event for the user.

20. The system of claim 17, wherein the sub-program for using the set of features that is periodically extracted from the data stream received from each of the mobile sensors to train the predictor to predict when an eating event for a user is about to occur comprises sub-programs for:
inputting the set of features that is periodically extracted from the data stream received from each of the mobile sensors into an overall set of features;
using a combination of a correlation-based feature selection method and a best-first decision tree machine learning method to select a subset of the features in the overall set of features; and
using the selected subset of the features to train the predictor to predict when an eating event for a user is about to occur.
